(19) United States
(12) Patent Application Publication
(10) Pub. No.: US 2023/0241198 A1
(43) Pub. Date: Aug. 3, 2023

(54) COMBINATION PORCINE VACCINE

(71) Applicant: BOEHRINGER INGELHEIM ANIMAL HEALTH USA INC., DULUTH, GA (US)

(72) Inventors: Edgar Diaz, Johns Creek, GA (US); Greg Stewart Cline, Plattsburg, MO (US); Jeremy Kroll, Urbandale, IA (US); Fernando Lopes Leivas Leite, Atlanta, GA (US); Michael B. Roof, Ames, IA (US); Mike Alan Steilen, St. Joseph, MO (US)

(21) Appl. No.: 18/003,056

(22) PCT Filed: Jul. 22, 2021
(86) PCT No.: PCT/US2021/042778

§ 371 Date: Dec. 22, 2022

Related U.S. Application Data
(60) Provisional application No. 63/055,947, filed on Jul. 24, 2020.

(30) Foreign Application Priority Data
Sep. 16, 2020 (EP) 20196349.3

(51) Int. Cl. A61K 39/02; A61K 39/295; A61K 39/39; A61P 31/04; A61P 31/20

(52) U.S. Cl. CPC A61K 39/105; A61K 39/39; A61K 39/0241; A61K 39/295; A61P 31/04; A61P 31/20; A61K 2039/70

(57) ABSTRACT

The present invention relates to a vaccine comprising an antigen of Lawsonia intracellularis and one or more antigens of at least one further pathogen selected from the group of porcine circovirus (PCV), Mycoplasma hyopneumoniae (M. hyo.) and porcine respiratory and reproductive syndrome virus (PRRSV), wherein the antigen of Lawsonia intracellularis is live Lawsonia intracellularis.

### RELATED APPLICATIONS AND INCORPORATION BY REFERENCE

Reference is made to PCT Publication Nos. WO 96/39629, WO 05/011731, WO 06/012949, WO 06/020730, WO 06/099561, WO 07/011993, WO 07/076520, WO 07/140244, WO 08/073464, WO 09/037262, WO 09/127684, WO 09/144088, WO 2011/054951, WO 2015/082457, WO 2015/082458, WO 2015/082465, WO 2016/124620, WO 2016/124623, WO 2017/068126, WO 2017/162741, WO 2018/189290, WO 2018/115435 and WO 2019/166362 and International Patent Application Serial No. PCT/US2020/026930 filed Apr. 6, 2020.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer’s instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

Disclosed herein is a combined porcine vaccine comprising a Lawsonia intracellularis antigen, a porcine circovirus (PCV) antigen, a Mycoplasma hyopneumoniae (M. hyo.) antigen, and a porcine respiratory and reproductive syndrome virus (PRRSV) antigen, methods of producing the same and uses thereof.

### BACKGROUND OF THE INVENTION

Lawsonia intracellularis, the causative agent of porcine proliferative enteropathy (“PPE”), affects virtually all animals, including humans, rabbits, ferrets, hamsters, fox, horses, and other animals as diverse as ostriches and emus and is a particularly great cause of losses in swine herds. A consistent feature of PPE is the occurrence of intracytoplasmic, non-membrane bound curved bacilli within enterocytes in affected portions of intestine. The bacteria associated with PPE have been referred to as “Campylobacter-like organisms.” S. McOrist et al., Vet. Pathol., Vol. 26, 260-64 (1989). Subsequently, the causative bacteria have been identified as a novel taxonomic genus and species, vernacularly referred to as Ileal symbiont (IS) intracellularis. C. Gebhart et al., Int′l. J. of Systemic Bacteriology, Vol. 43, No. 3, 533-38 (1993). These novel bacteria have been given the taxonomic name Lawsonia (L.) intracellularis. S. McOrist et al., Int′l. J. of Systemic Bacteriology, Vol. 45, No. 4, 820-25 (1995). These three names have been used interchangeably to refer to the same organism as further identified and described herein.

Porcine circovirus type (PCV) is a non-enveloped, icosahedral single-stranded DNA (ssDNA) virus belonging to the genus Circovirus in the family Circoviridae. The genome encodes for two major open reading frames (ORFs) where ORF1 encodes a replication-associated protein (rep) and ORF2 encodes the viral capsid (cap) protein, which determines the antigenic characteristics of the virus. PCV2 shares approximately 80% sequence identity with porcine circovirus type 1 (PCV1). However, in contrast with PCV1, which is generally non-virulent, swine infected with PCV2 exhibit a syndrome commonly referred to as Post-weaning Multisystemic Wasting Syndrome (PMWS). PCV3 is genetically distinct from porcine circovirus type 2 (PCV2); specifically, there is only 48% amino acid identity in the rep gene and 26% amino acid identity in the cap gene between the two viruses.

Mycoplasma hyopneumoniae (M. hyo.) is a small bacterium (400-1200 nm) classified in the mycoplasmataceae family. M. hyo. is associated with Enzootic Pneumonia, a swine respiratory disease commonly seen in growing and finishing pigs. M. hyo. attacks the cilia of epithelial cells of the windpipe and lungs, causing the cilia to stop beating (ciliostasis) and eventually causing areas of the lungs to collapse. M. hyo. is considered to be a primary pathogen that facilitates entry of PRRSV and other respiratory pathogens into the lungs. Separate strains, 232, J & 7448, have had their genomes sequenced (Minion et al., J. Bacteriol. 186:7123-33, 2004; Vasconcelos et al., J. Bacteriol. 187:5568-77, 2005 and Han et al., Genome Announc. 2017 Sep; 5(38): e01012-17). Porcine reproductive and respiratory syndrome (PRRS) is viewed by many as the most important disease currently affecting the pig industry worldwide. PRRS virus (PRRSV) is an enveloped single stranded RNA virus classified in the family Arteriviridae. There is large variability in the antigenic characteristics of the different isolates of PRRSV and effective measures to prevent infections are limited. There are three major groups of vaccines available for PRRS, attenuated modified live virus (MLV), killed virus vaccine or recombinant vaccines. The viral envelope proteins of PRRSV are generally categorized into major and minor proteins based on abundance of proteins in the virion. The major viral envelope proteins are gp5 (ORF 5) and M (ORF 6) and form a dimer. The minor envelope proteins are gp2 (ORF2), gp3 (ORF3), gp4 (ORF4) and E (ORF2b) and probably a newly identified viral protein gp5a (ORF 5a). The active antigenic component can include the ORF4, ORF5, ORF6, or ORF7 from PRRSV virus.

There is an ongoing need for new modes of immunizing animals against the above pathogens.

Thus, the technical problem underlying the present invention is the provision of further means and methods for immunizing animals against pathogens. The problem is solved and the above-mentioned needs are addressed by the provision of the embodiments characterized in the claims and as provided herein below.

Citation or identification of any document in this application is not an admission that such document is available as prior art to the present invention.

### SUMMARY OF THE INVENTION

The present invention relates to a vaccine comprising an antigen of Lawsonia intracellularis and one or more antigens of at least one further pathogen selected from the group of porcine circovirus (PCV), Mycoplasma hyopneumoniae (M. hyo.) and porcine respiratory and reproductive syndrome virus (PRRSV), wherein the antigen of Lawsonia intracellularis is live Lawsonia intracellularis. Accordingly, the vaccine may comprise live Lawsonia intracellularis and an antigen of PCV. Thus, the vaccine may comprise live Lawsonia intracellularis and a PCV2 ORF2 protein.

The vaccine may also comprise live Lawsonia intracellularis and an antigen of M. hyo..

Thus, the vaccine may comprise live Lawsonia intracellularis and a M. hyo. bacterin.

The vaccine may also comprise live Lawsonia intracellularis and an antigen of PRRSV.

Thus, the vaccine may comprise live Lawsonia intracellularis and an attenuated PRRSV virus.

The vaccine may also comprise live Lawsonia intracellularis, an antigen of PCV and an antigen of M. hyo..

Thus, the vaccine may comprise live Lawsonia intracellularis and a PCV2 ORF2 protein and a M. hyo. bacterin.

The vaccine may also comprise live Lawsonia intracellularis, an antigen of PCV and an antigen of PRRSV.

Thus, the vaccine may comprise live Lawsonia intracellularis and a PCV2 ORF2 protein and an attenuated PRRSV virus.

The vaccine may also comprise live Lawsonia intracellularis, an antigen of PRRSV and an antigen of M. hyo..

Thus, the vaccine may comprise live Lawsonia intracellularis and an attenuated PRRSV virus and a M. hyo. bacterin.

The vaccine may also comprise live Lawsonia intracellularis an antigen of PCV, an antigen of M. hyo. and an antigen of PRRS.

Thus, the vaccine may comprise live Lawsonia intracellularis and a PCV2 ORF2 protein and M. hyo. bacterin and an attenuated PRRSV virus.

Preferably, the vaccine comprises live Lawsonia intracellularis and an antigen of PCV.

More preferably, the vaccine comprises live Lawsonia intracellularis and a PCV2 antigen. More preferably, the vaccine comprises live Lawsonia intracellularis and a recombinant polypeptide of PCV2.

In a particularly preferred embodiment, the vaccine comprises the antigen of Lawsonia intracellularis included in Enterisol® Ileitis and the PCV antigen included in Ingelvac CircoFLEX® or 3FLEX®. The term “live Lawsonia intracellularis” includes “modified-live Lawsonia intracellularis” and “attenuated Lawsonia intracellularis”.

The vaccine of the present invention may have a dosage of Lawsonia intracellularis of about 10³ to 10⁹ bacteria/Kg of body weight, preferably of about 10⁵ to 10⁷ bacteria/Kg of body weight. The vaccine of the present invention may also have a dosage of the antigen of Lawsonia intracellularis of about 10⁵ to about 10⁷ of Lawsonia intracellularis bacteria.

The antigen of Lawsonia intracellularis may be lyophilized. Preferably, the antigen of Lawsonia intracellularis is the antigen included in Enterisol® Ileitis.

The PCV antigen of the vaccine of the present invention may be an antigen of PCV1, PCV2 or PCV3. Preferably, the PCV antigen is a PCV2 antigen. The PCV antigen may be a recombinant polypeptide. Said recombinant polypeptide may be expressed by a PCV ORF gene. Preferably, the PCV ORF gene is a PCV ORF2 gene. The PCV recombinant polypeptide may be expressed in a baculovirus cell. Preferably, the PCV antigen is the antigen included in Ingelvac CircoFLEX® or the antigen of PCV included 3FLEX®. In the vaccine of the present invention the antigen of PCV may have a dosage of about 2 µg to about 400 µg.

The antigen of M. hyo. of the vaccine of the present invention may be a supernatant and/or a bacterin. The detailed description of a supernatant and bacterin is provided herein below. Preferably, the antigen of M. hyo. is the antigen of M. hyo. included in Ingelvac MycoFLEX® or the antigen of M. hyo. included in 3FLEX®.

The PRRSV antigen of the vaccine of the present invention may be a live PRRSV virus. Said live virus may be a modified and/or an attenuated virus. The vaccine of the present invention may have a dosage of the antigen of PRRSV of about 10¹ to about 10⁷ viral particles per dose, preferably about 10³ to about 10⁵ particles per dose, more preferably about 10⁴ to about 10⁵ particles per dose. The vaccine of the present invention may have a dosage of the antigen of PRRSV of about 10⁴ to about 10⁷ viral particles per dose. The antigen of PRRSV may be lyophilized. Preferably, the PRRSV antigen is the PRRSV antigen included in Ingelvac® PRRSV MLV or the PRRSV antigen included in 3FLEX®.

The antigen of PCV, the antigen of M. hyo. and the antigen of PRRSV may be the antigens included in 3FLEX®.

The vaccine of the present invention may be lyophilized antigen of Lawsonia intracellularis dissolved in 3FLEX®. Accordingly, the vaccine of the present invention may be lyophilized live Lawsonia intracellularis dissolved in 3FLEX®. Furthermore, the vaccine of the present invention may be Enterisol® Ileitis dissolved in 3FLEX®.

In one embodiment, the vaccine of the invention may further comprise a pharmaceutically or veterinarily acceptable carrier. In one embodiment, the vaccine of the invention may further comprise one or more adjuvant(s). Suitable adjuvants are known in the art and non-limiting examples are described herein. The vaccine of the present invention may comprise as an adjuvant one or more of a polymer of acrylic or methacrylic acid; a copolymer of maleic anhydride and an alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol®; Carbopol® 974P; Carbopol® 934P; Carbopol® 971P; Carbopol® 980; Carbopol® 941P; ImpranFLEX®; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

Preferably, the adjuvant is a carbomer. Advantageously, the adjuvant may be ImpranFLEX® and/or Carbopol®.

The vaccine of the present invention may be in a form for systemic administration.

The vaccine of the present invention may be formulated and/or packaged for a single dose or one-shot administration.

The vaccine of the present invention may be formulated and/or packaged for a multi-dose regimen, preferably a two-dose regimen.

The vaccine of the present invention may be in a dosage form, wherein said dosage form is delivered from a container containing a larger amount of said vaccine and wherein a dosage form of said vaccine is capable of being delivered from said container. Said container may contain at least 10, at least 50, at least 100, at least 150, at least 200 or at least 250 doses of said vaccine.

The present invention also encompasses the vaccine of the present invention for use in a method for eliciting a protective immune response in an animal comprising administering said vaccine to the animal.

The present invention also encompasses the vaccine of the present invention for use in a method for eliciting a protective immune response in a pig comprising administering said vaccine to the pig.

The present invention also encompasses the vaccine of the present invention for use in a method for eliciting a protective immune response against Lawsonia intracellularis and/or PCV and/or M. hyo. and/or PRRSV in the animal.

In a preferred embodiment the vaccine of the present invention is for use in a method for eliciting a protective immune response against Lawsonia intracellularis and PCV.

In a preferred embodiment the vaccine of the present invention is for use in a method for eliciting a protective immune response against Lawsonia intracellularis and M. hyo..

In a preferred embodiment the vaccine of the present invention is for use in a method for eliciting a protective immune response against Lawsonia intracellularis and PRRS.

In a preferred embodiment the vaccine of the present invention is for use in a method for eliciting a protective immune response against Lawsonia intracellularis and PCV and M. hyo..

In a preferred embodiment the vaccine of the present invention is for use in a method for eliciting a protective immune response against Lawsonia intracellularis and PCV and PRRS.

In a preferred embodiment the vaccine of the present invention is for use in a method for eliciting a protective immune response against Lawsonia intracellularis and PRRS and M. hyo..

In a preferred embodiment the vaccine of the present invention is for use in a method for eliciting a protective immune response against Lawsonia intracellularis and PCV and M. hyo. and PRRSV. The present invention also encompasses the vaccine of the present invention for use in a method for eliciting a protective immune response, wherein the vaccine is administered systemically.

The present invention also encompasses the vaccine of the present invention for use in a method for eliciting a protective immune response, wherein the vaccine is administered as one dose.

The present invention also encompasses the vaccine of the present invention for use in a method for eliciting a protective immune response, wherein the vaccine is administered as at least one dose.

The present invention also encompasses the vaccine of the present invention for use in a method for eliciting a protective immune response, wherein said animal is simultaneously/concomitantly treated with one or more antibiotic(s).

The present invention also encompasses the vaccine of the present invention for use in a method for immunizing an animal against a clinical disease caused by at least one pathogen in said animal, wherein said vaccine fails to cause clinical signs of infection but is capable of inducing an immune response that immunizes the animal against pathogenic forms of said at least one pathogen.

The present invention also encompasses the vaccine of the present invention for use in a method for eliciting a protective immune response, wherein the protective immune response against Lawsonia intracellularis is for reducing intestinal lesions in an animal, in comparison to an animal of a non-immunized control group of the same species. Thus, the vaccine of the present invention is for use in a method for reducing intestinal lesions in an animal, in comparison to an animal of a non-immunized control group of the same species, comprising administering to the animal said vaccine. The intestinal lesions may be ileum lesions.

The intestinal lesions and/or ileum lesions may be macroscopic lesions and/or microscopic lesions. The present invention also encompasses the vaccine of the present invention for use in a method for eliciting a protective immune response, wherein the protective immune response against Lawsonia intracellularis is for reducing fecal shedding of an animal, in comparison to an animal of a non-immunized control group of the same species. Thus, the vaccine of the present invention is for use in a method for reducing fecal shedding of an animal, in comparison to an animal of a non-immunized control group of the same species, comprising administering to the animal said vaccine.

The present invention also encompasses the vaccine of the present invention for use in a method for eliciting a protective immune response, wherein the protective immune response against Lawsonia intracellularis is for increasing the average daily weight gain of an animal, in comparison to an animal of a non-immunized control group of the same species. Thus, the vaccine of the present invention is for use in a method for increasing the average daily weight gain of an animal, in comparison to an animal of a non-immunized control group of the same species, comprising administering to the animal said vaccine.

The present invention also encompasses the vaccine of the present invention for use in a method for eliciting a protective immune response, wherein the vaccine is protective against a challenge with 8×10⁹ Lawsonia bacteria.

The present invention also encompasses a method for eliciting an immune response or an immunological response or a protective immune or immunological response against Lawsonia intracellularis, PCV, M. hyo. and PRRSV in an animal which may comprise administering to the animal any of the herein disclosed vaccines.

The present invention also encompasses a method of immunizing an animal against a clinical disease caused by at least one pathogen in said animal, wherein said method may comprise the step of administering to the animal the vaccine of any one of the herein disclosed vaccines, wherein said vaccine fails to cause clinical signs of infection but is capable of inducing an immune response that immunizes the animal against pathogenic forms of said at least one pathogen.

Accordingly, the present invention encompasses the use of a vaccine of the present invention in the preparation of a composition for inducing a protective immune response against Lawsonia intracellularis and/or PCV and/or M. hyo. and/or PRRSV or for a method for inducing a protective immune response against Lawsonia intracellularis and/or PCV and/or M. hyo. and/or PRRSV.

The present invention also encompasses the vaccine of the present invention for use in a method for eliciting a protective immune response in an animal comprising administering said vaccine to the animal, wherein the animal is simultaneously/concomitantly treated with one or more antibiotic(s).

Furthermore, the present invention encompasses the vaccine of the present invention for use in a method for eliciting a protective immune response in an animal comprising administering said vaccine to the animal, wherein the animal is simultaneously/concomitantly treated with one or more antibiotic(s), and wherein said vaccine comprises live Lawsonia intracellularis and/or a PCV2 ORF2 protein and/or a M. hyo bacterin and/or an attenuated PRRSV virus.

Further, the present invention encompasses the vaccine of the present invention for use in a method for eliciting a protective immune response in an animal comprising administering said vaccine to the animal, wherein the animal is simultaneously/concomitantly treated with one or more antibiotic(s), and wherein said vaccine comprises live Lawsonia intracellularis and a PCV2 ORF2 protein and a M. hyo bacterin and an attenuated PRRSV virus.

The present invention encompasses the vaccine of the present invention for use in a method for eliciting a protective immune response in an animal comprising administering said vaccine to the animal, wherein the animal is simultaneously/concomitantly treated with one or more antibiotic(s), and wherein said vaccine comprises live Lawsonia intracellularis and a PCV2 ORF2 protein.

The present invention encompasses the vaccine of the present invention for use in a method for eliciting a protective immune response in an animal comprising administering said vaccine to the animal, wherein the animal is simultaneously/concomitantly treated with one or more antibiotic(s), and wherein said vaccine comprises the antigen of Lawsonia intracellularis included in Enterisol® Ileitis and the antigen of PCV included Ingelvac CircoFLEX® or 3FLEX®.

In addition, the present invention encompasses the vaccine of the present invention for use in a method for eliciting a protective immune response in an animal comprising administering said vaccine to the animal, wherein the animal is simultaneously/concomitantly treated with Denagard® (tiamulin) and/or CTC (chlortetracycline), and wherein said vaccine comprises the antigen of Lawsonia intracellularis included in Enterisol® Ileitis and the antigen of PCV included Ingelvac CircoFLEX® or 3FLEX®.

It is further noted that the invention does not intend to encompass within the scope of the invention any product, process, or making of the product or method of using the product, which does not meet the written description and enablement requirements of the USPTO (35 U.S.C. §112, first paragraph), such that Applicants reserve the right and hereby disclose a disclaimer of any previously described product, process of making the product, or method of using the product. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows: Average Ileum Gross Lesion Scores. Different letters indicate statistical significance (p<0.05), error bars represent standard error of the mean.

FIG. 2 shows: Average Ileum Gross Lesion Length. Different letters indicate statistical significance (p<0.05), error bars represent standard error.

FIG. 3 shows: Average Ileum Lesion Severity. Different letters indicate statistical significance (p<0.05), error bars represent standard error.

FIG. 4 shows: Group Average Daily Weight Gain (lbs). Different letters indicate statistical significance (p<0.05), error bars represent standard error.

FIG. 5 shows: Percentage of animals with serum ELISA positive results for Lawsonia.

FIG. 6 shows: Average quantity of L. intracellularis shed by day. Different letters indicate statistical significance (p<0.05), error bars represent standard error.

FIG. 7 shows: Average microscopic lesion scores measured in the terminal ileum. Different letters indicate statistical significance (p<0.05), error bars represent standard error.

FIG. 8 shows: Average immunohistochemistry score for the presence of L. intracellularis antigen in ileum tissue.

FIG. 9 shows: Vaccine blending.

FIG. 10 shows: Study Outline. Tiamulin/CTC given only to the EIIMATB group in feed, one week prior to and following vaccination. Both groups (EIIM and EIIMATB) received vaccine at the same time, 4 weeks prior to challenge. All animals were euthanized and necropsied at 21 days post infection (dpi)* = blood and fecal collection. Clinical scoring every day 0-21.

### DETAILED DESCRIPTION OF THE INVENTION

The present invention relates to a vaccine comprising an antigen of Lawsonia intracellularis and one or more antigens of at least one further pathogen selected from the group of porcine circovirus (PCV), Mycoplasma hyopneumoniae (M. hyo.) and porcine respiratory and reproductive syndrome virus (PRRSV), wherein the antigen of Lawsonia intracellularis is live Lawsonia intracellularis. In one embodiment the vaccine of the present invention comprises live Lawsonia intracellularis and an antigen of PCV.

In one embodiment the vaccine of the present invention comprises live Lawsonia intracellularis and a PCV2 ORF2 protein.

In one embodiment the vaccine of the present invention comprises live Lawsonia intracellularis and an antigen of M. hyo.

In one embodiment the vaccine of the present invention comprises live Lawsonia intracellularis and a M. hyo bacterin.

In one embodiment the vaccine of the present invention comprises live Lawsonia intracellularis and an antigen of PRRSV.

In one embodiment the vaccine of the present invention comprises live Lawsonia intracellularis and an attenuated PRRSV virus.

In one embodiment the vaccine of the present invention comprises live Lawsonia intracellularis, an antigen of PCV and an antigen of M. hyo.

In one embodiment the vaccine of the present invention comprises live Lawsonia intracellularis and a PCV2 ORF2 protein and a M. hyo bacterin.

In one embodiment the vaccine of the present invention comprises live Lawsonia intracellularis, an antigen of PCV and an antigen of PRRSV.

In one embodiment the vaccine of the present invention comprises live Lawsonia intracellularis and PCV2 ORF2 protein and an attenuated PRRSV virus.

In one embodiment the vaccine of the present invention comprises live Lawsonia intracellularis, an antigen of PRRSV and an antigen of M. hyo.

In one embodiment the vaccine of the present invention comprises live Lawsonia intracellularis and an attenuated PRRSV virus and a M. hyo bacterin.

In one embodiment the vaccine of the present invention comprises live Lawsonia intracellularis, an antigen of PCV, an antigen of M. hyo and an antigen of PRRSV.

In one embodiment the vaccine of the present invention comprises live Lawsonia intracellularis and PCV2 ORF2 protein and M. hyo bacterin and an attenuated PRRSV virus.

With respect to the components of the vaccine of the invention, it is noted that the terms “vaccine” and “antigen” are sometimes used synonymously herein. Accordingly, “vaccine comprises a PCV vaccine” may for example be used synonymously with “vaccine comprises a PCV antigen”. With respect to the vaccine of the invention, the terms “vaccine” and “immunogenic composition” may be used synonymously herein.

The terms “antigen”, “immunogen” and “immunogenic component” may be used synonymously herein.

The terms “immune response”, “immunological response”, “protective immune response” and “protective immunological response” may be used synonymously herein.

Furthermore, it is noted that all disclosures provided in this specification can be combined. Thus, for example a specific disclosure provided herein in connection with a PCV vaccine or antigen can also be combined with a PRRSV vaccine or antigen and vice versa, provided that such transfer would be considered feasible by a person skilled in the art based on the teaching herein. In other words, when a method, technique, administration route etc. is disclosed in connection with, e.g., PCV, it may not be limited to PCV but it can also be used in connection with, e.g., PRRSV. Furthermore, all disclosures provided herein for a certain described vaccine comprising a single antigen may also be applied to a described vaccine comprising more than one antigen.

The disclosures in the context of the methods of the invention described herein are applicable to the corresponding uses and vice versa.

The vaccine of the present invention comprises an antigen of Lawsonia intracellularis. Accordingly, an immunogenic composition for eliciting a protective immune response in a pig against Lawsonia intracellularis is provided.

As used herein, the term “Lawsonia intracellularis” or “L. intracellularis” means the intracellular, curved, gram-negative bacteria described in detail by C. Gebhart et al., Int′l. J. of Systemic Bacteriology, Vol. 43, No. 3, 533-38 (1993) and S. McOrist et al. Int′l. J. of Systemic Bacteriology, Vol. 45, No. 4, 820-25 (1995) (each of which is incorporated herein by reference in its entirety) and includes, but is not limited to, the bacteria deposited as ATCC 55672 in the American Type Culture Collection, Rockville, Md; the bacteria deposited as NCTC 12656 and 12657 in the National Collection of Type Cultures, Colindale, London; the causative bacteria which can be obtained from PPE infected swine or other animals throughout the world given the knowledge in the art and the teachings herein; and variants or mutants of any of the above bacteria, whether spontaneously or artificially obtained.

“Live L. intracellularis” as used herein means that the L. intracellularis bacteria are live bacteria. WO 96/39629 and WO 05/011731 describe non-pathogenic live or attenuated strains of L. intracellularis. However, the vaccine composition of the invention as described herein may comprise inactivated/killed L. intracellularis bacteria due to the production/formulation step. As used herein, the term “attenuated strain” means any L. intracellularis strain that is prepared according to cultivation and passaging techniques known in the art and/or as taught herein to achieve a reduced virulence, preferably avirulence, while maintaining immunogenic properties when administered to a host animal. As demonstrated below, various different L. intracellularis strains have been cultivated and attenuated according to the present teachings to obtain attenuated immunogenic strains having efficacy as vaccines in swine and other animals susceptible to L. intracellularis infection.

A genetically modified virus and/or bacterium or a modified-live virus and/or bacterium is “attenuated” if it is less virulent than its unmodified parental strain. A strain is “less virulent” if it shows a statistically significant decrease in one or more parameters determining disease severity. Such parameters may include level of viremia, bacteremia, fever, severity of respiratory distress, severity of reproductive symptoms, or number or severity of lesions in an organ, such as the intestine (particularly the ileum) or the lung, etc.

The attenuated strains for use in the vaccine of the invention are expected to have utility as immunogens in antimicrobial vaccines for animals, including birds, fish, cattle, swine, horses, mammals and primates in general, and humans. Such vaccines can be prepared by techniques known to those skilled in the art, given the teachings contained herein. Such a vaccine would comprise an immunologically effective amount of the attenuated strain in a pharmaceutically acceptable carrier. The vaccine could be administered in one or more doses. An immunologically effective amount is determinable by means known in the art without undue experimentation, given the teachings contained herein. The amount of avirulent bacteria should be sufficient to stimulate an immune response in disease-susceptible animals while still being avirulent. This will depend upon the particular animal, bacteria, and disease involved. The recommended dose to be administered to the susceptible animal is preferably about 10³ to 10⁹ bacteria/Kg of body weight and most preferably about 10⁵ to 10⁷ bacteria/Kg of body weight. The carriers are known to those skilled in the art and include stabilizers and diluents. Such a vaccine may also contain an appropriate adjuvant. The vaccine of the invention may be used in combination with other vaccines, for example, as a diluent of another lyophilized vaccine, or combined before lyophilization with another vaccine or simply mixed together. In another embodiment, the mixture of two or more liquid vaccines is also contemplated. The vaccine preparations may also be desiccated, for example, by freeze drying for storage purposes or for subsequent formulation into liquid vaccines.

Accordingly, the invention also comprises a method for inducing an immune response to virulent, wild-type L. intracellularis bacteria in an animal host for the purpose of protecting the host from such bacteria. The method comprises administering an immunologically effective amount of the live, modified-live or attenuated bacteria or bacteria described herein to the host and, preferably, administering the vaccine of the invention to the host.

As used herein, the term “large-scale cultivation” means a level of cultivation of L. intracellularis greater than approximately 2.0 to 3.0 liters and includes production on a scale of 100 liters or more. “Cultivation” as used herein, means the process of promoting the growth, reproduction and/or proliferation of L. intracellularis.

In practicing the method for cultivation of the herein described bacteria culture cells may first be inoculated with an inoculum comprising L. intracellularis bacteria so as to infect the cells with the bacteria. Numerous cell lines can be used in practicing the invention, including, but not limited to, IEC-18 (ATCC 1589)--rat intestinal epithelial cells, HEp-2 (ATCC 23)--human epidermoid carcinoma cells, McCoy (ATCC 1696)--mouse (nonspecified) cells, MDCK (ATCC 34)--Madin-Darby canine kidney cells, BGMK (Biowhittaker #71-176)--buffalo green monkey kidney cells, and swine intestinal epithelium cells. The preferred culture cells are HEp-2, McCoy or IEC-18 cells. Alternatively, the bacteria may be cultivated in a cell free system so long as the bacteria are maintained at the appropriate dissolved O₂ concentration as taught herein.

If culture cells are used, prior to being inoculated, the cells are preferably but need not be in the form of a monolayer. To form a monolayer, the cells may be seeded into conventional flasks. Each flask is generally seeded with between about 1 × 10⁵ cells to about 10 × 10⁵ cells per 25 cm² flask mixed with growth media. The growth media may be any media for cell cultivation which includes a nitrogen source, necessary growing factors for the chosen culture cells, and a carbon source, such as glucose or lactose. The preferred media is DMEM with 2-5% fetal bovine serum, although various other commercially available media may be used with good results.

Applicants have found that successful cultivation of L. intracellularis is enhanced by maintaining the culture cells in a constant state of growth. Therefore, the culture cell monolayer should be at about 20 percent to about 50 percent confluency at the time of inoculation. Preferably, the cells should be at about 30 percent to about 40 percent confluency at the time of inoculation, with about 30 percent confluency being most preferred.

The inoculum may be a pure culture of L. intracellularis obtained, for example, from ATCC deposit 55672, NCTC deposits 12656 or 12657, or from infected swine or other animals using the isolation and purification teachings discussed herein.

According to one embodiment, the inoculum for practicing the invention is an intestinal homogenate prepared by scraping the mucosa off of the ileum of a swine or other animal infected with PPE. When preparing an intestinal homogenate, ileal sections selected for culture should show severe lesions with gross thickening of the gut. Due to the fragile nature of the bacteria, samples should preferably be stored at -70° C. as quickly as possible after necropsy. An antibiotic to which L. intracellularis is resistant such as Vancomycin, Amphotericin B or members of the aminoglycoside group of antibiotics, including Gentamicin and Neomycin, to name a few, is preferably added to the inoculum to suppress contaminating bacteria while permitting L. intracellularis growth. Whether the inoculum is a pure culture or an intestinal homogenate, inoculation of the culture cells can be performed by various techniques known in the art given the teachings herein.

The bacteria and/or inoculated culture cells are then incubated under a reduced dissolved O₂ concentration. At dissolved oxygen concentrations greater than 18% L. intracellularis growth is less than optimal with cessation of growth eventually occurring at oxygen concentrations outside this range. Preferably, the inoculated culture cells are incubated in a dissolved oxygen concentration in the range of from about 0% to about 10%. More preferably, the cells are incubated in an oxygen concentration in the range of from about 0% to about 8%, with an oxygen concentration of about 0% to about 3.0% being most preferred.

The proper concentration of carbon dioxide is also important to the proper growth of L. intracellularis. At carbon dioxide concentrations greater than 10% and less than 4%, non-optimum growth occurs with cessation of growth eventually occurring at carbon dioxide concentrations outside this range. Preferably, the carbon dioxide concentration is in the range from about 6% to about 9%, with a carbon dioxide concentration of about 8.8% being most preferred.

In addition, the cells are preferably incubated at a hydrogen concentration in the range from about 73% to about 94%. Nitrogen may be used in place of some or all of the hydrogen present. According to a particularly preferred embodiment, the cells are incubated in about 0-8.0% O₂, about 8.8% CO₂, and about 83.2% H₂.

Inoculated cells may be incubated in a dual gas incubator or other gas chamber which contains the proper oxygen and carbon dioxide concentrations and which allows the cells to be suspended during incubation. The chamber should comprise a means for maintaining the inoculated cells in suspension, and a gas monitor and supply source to supply and maintain the proper gas concentrations. The incubation temperature should be in the range of from 30° C. to 45° C. and is more preferably in the range of from about 36° C. to about 38° C. Most preferably, the temperature is about 37° C. The necessary equipment for the cultivation and attenuation methods of the invention is readily available to those of ordinary skill in the art given the teachings herein. One example of equipment suitable for carrying out the present invention is a dual gas incubator, e.g., model 480 available from Lab-Line, Melrose Park, Ill., in conjunction with spinner flasks to maintain the cells in suspension. The presently preferred equipment comprises a fermentor, bioreactor or rotary shaker containing at least about 2 litres media and capable of maintaining the culture cells in suspension via sparging gas of the appropriate concentration, or other means of mechanical agitation, and continuously monitoring dissolved O₂ levels in the media. New Brunswick, Braun and other companies make suitable fermentors and bioreactors for this purpose. By maintaining the inoculated cells in a suspended state during incubation, maximum growth of the cells, and hence L. intracellularis, is achieved by increasing each individual cell’s exposure to growth media and the proper mixture of oxygen and carbon dioxide. The culture cells can be agitated and maintained in suspension by a variety of methods known in the art, including, for example, culture flasks, roller bottles, membrane cultures and spinner flasks. The cells may be kept in suspension during incubation by incubating the cells in a spinner flask inside a dual gas incubator or similar apparatus. The term “spinner flask”, as used herein, means a flask or other container which employs a paddle, propeller or other means to agitate the culture and keep the cells contained therein in suspension.

In a particularly preferred embodiment of the invention, the inoculated cells are incubated until the cells reach confluency and then the cells are placed in a spinner flask containing growth media and incubated in a dual gas incubator while spinning the flask. Preferably, the inoculated cells are scraped into the spinner flask. This can be achieved by a variety of methods known in the art such as using a cell scraper to detach the cells. Once the cells are introduced into the spinner flask, the paddle of the spinner flask is typically rotated in the range of from about 30 to about 60 rpm in order to maintain the infected cells in suspension.

A portion of the cultivated L. intracellularis is then passaged to fresh culture cells to increase the production of L. intracellularis bacteria. The term “passaging” or variations thereof herein means the process of transferring a portion of the cultivated L. intracellularis to fresh culture cells in order to infect the fresh cells with the bacterium. The term “fresh”, as used herein, means cells which have not yet been infected by L. intracellularis. Preferably such cells are, on the average, no more than approximately one day old.

The passage of L. intracellularis in suspension cultures may be accomplished by removing a portion of the original culture and adding it to a new flask containing fresh culture cells. If the original culture has a high number of bacteria/ml, for example, greater than about 10⁴ bacterial/ml, it is preferable to add between about 1 to 10% (volume to volume) of culture from the infected flask to a new flask containing fresh cells. This is preferably done when 50-100% of the cells are infected. If fewer than 50% of the cells are infected, passaging is preferably accomplished by splitting the culture 1:2 into a new flask and scaling-up the volume by adding fresh media. In either case, cell lysis and other steps are not required, in direct contrast to the passage of monolayer cultures, as in the prior art.

After sufficient growth of the culture cells and subsequent infection by L. intracellularis at greater than about 70% cell infectivity, as determined by IFA, TCID₅₀ or other comparable method, at least a portion of the cultivated L. intracellularis bacteria is then harvested. However, in case different results are achieved using different techniques for determining cell infectivity, the results of the IFA method shall be used. The harvesting step may be performed by separating the bacteria from the suspension by various techniques known to those of ordinary skill in the art, given the teachings herein. Preferably, the L. intracellularis bacteria is harvested by centrifuging the contents of all or a portion of the suspension to pellet the culture cells, resuspending the resulting cell pellets, and lysing the infected cells. Typically, at least a portion of the contents is centrifuged at about 3000 × g for about 20 minutes in order to pellet the cells and bacteria. The pellet may then be resuspended in, for example, a sucrose-phosphate-glutamate (SPG) solution and passed approximately four times through a 25 gauge needle in order to lyse the cells. If further purification is desired, the samples can be centrifuged at about 145 × g for about five minutes to remove cellular nuclei and debris. The supernatant may then be centrifuged at about 3000 × g for about twenty minutes and the resulting pellet resuspended in an appropriate diluent, such as SPG with fetal bovine serum (to prepare harvested bacteria suitable for freezing or use as an inoculant) or growth media (to prepare harvested bacteria more suitable for passaging to fresh cells).

As previously mentioned, effective growth of L. intracellularis for large-scale production is enhanced by keeping the tissue cells actively growing. With monolayers, when cultures become confluent the rate of cell division decreases substantially. Attempts to grow L. intracellularis on monolayer tissue cultures have had limited success and scale-up has not been possible. However, using suspension cultures greatly facilitates keeping the cells actively growing and permits continuous culture expansion and scale-up. Using a fermentor and between about 0-3% dissolved

O₂ as explained above, Applicants have been able to grow up to 10⁸ bacteria/ml. Applicants have also been able to keep the cultured bacteria actively growing for many months and expect to be able to do so indefinitely.

Previously, it was generally believed that cells must be attached to a surface in order to be infected by L. intracellularis. The cell suspensions disclosed herein are unique and contradict this theory. When using McCoy or IEC-18 cells, one may add gelatin, agarose, collagen, acrylamide or silica beads, such as Cultisphere-G porous microcarriers manufactured by HyClone Laboratories, Logan, Utah, along with the growth media. In one embodiment, uninfected McCoy cells may be added to the media during cell culture growth of McCoy cells infected by L. intracellularis. However, McCoy cells as well as HEp-2 cells may be used in the cultivation method of the invention without requiring microcarriers. This provides an especially advantageous and economical route for large-scale cultivation.

For culture maintenance purposes, with HEp-2 cultures, preferably 25-50% of the culture is removed and replaced with fresh media at weekly intervals. For cell cultures with microcarriers or beads, preferably 25-50% of the culture is removed and replaced with fresh microcarriers or beads and fresh media 1-2 times weekly. For scale-up purposes, an additional 25-50% of media, or media with microcarriers, may be added to the culture.

Depending upon the rate at which the culture cells become infected, passage to fresh cells generally occurs between about every 2 to about 5 weeks. Assuming that the culture cells become at least 70% infected within 2-3 weeks, preferably passage occurs between about every 3 to 4 weeks.

Live L. intracellularis antigen for use in the vaccine of the invention may be produced in accordance with the above-outlined production methods. According to a particularly preferred embodiment, after maintaining the infected cells in suspension for an extended time (for example, 6-8 months), at least a portion of the cultivated L. intracellularis bacteria are harvested and monitored for potential attenuation. Such monitoring is preferably accomplished by host animal or animal model challenges to select for an attenuated strain. Such attenuated strains are used in vaccines according to the methods taught herein. The attenuated L. intracellularis vaccines according to the present invention have shown efficacy against L. intracellularis infection in a variety of animals and are expected to be effective in humans as well.

Cultivation in suspension allows rapid culture expansion, an increase in yields of 100-1000 fold, and reduced cost. As a result, the abundant supply of L. intracellularis bacteria produced according to the herein disclosed cultivation method is readily attenuated for vaccine production purposes. Attenuation is difficult in monolayer cultures due to the low yield of bacteria produced using conventional monolayer growing techniques. In contrast, the disclosed method of growing L. intracellularis greatly increases the ease, speed, and number of bacterium available for this purpose. The more cells and cell divisions which occur, the greater the level of mutations occurring which are advantageous in vaccine development. Growth in suspensions increases the expression of important immunogens controlled by environmentally regulated genes and their expression products.

The resulting attenuated strains can be cultivated in tissue culture monolayers as described in Example 1 of U.S. Pat. No. 5,885,823, but are preferably cultivated in suspension cultures according to the method disclosed herein. Other means of attenuation can include chemical attenuation by the use of, for example, N-methyl nitrosoguanadine and others known in the art. Whether by multiple passage or chemical means, an attenuated L. intracellularis is produced and selected for vaccine preparation.

According to one vaccine embodiment of the invention, the antigen is harvested by centrifugation or microfiltration as described above. The antigen is then standardized at a defined level based on the optimum host animal immune response, determined by a dose titration in the host animal species.

According to a particularly preferred vaccine embodiment using the cultivation methods previously described, the bacteria are serially passaged to induce and select for an attenuated, avirulent live culture. The culture is tested in the host animal (after preferably at least 6 to 8 months or more of growth in the suspension culture) for signs of attenuation. The culture is harvested as described earlier and diluted. Pigs, for example, may be orally vaccinated with at least 1 × 10⁵ to 1 × 10⁶ bacteria. About twenty-eight days after vaccination, the pigs are orally inoculated with about 1 ×10⁷ organisms from a less passaged (about 30 to 45 days old) virulent culture of L. intracellularis. The infected animals are necropsied 21 days after challenge and the small intestines observed for gross lesions as well as microscopic lesions. PCR should also be performed. About eighty percent of the control animals will show gross or microscopic lesions and test positive for the presence of L. intracellularis in the mucosal cells of the intestines using either PCR or FA testing methods. Vaccinated animals will have normal mucosal surfaces as determined by histological observations and will be negative by PCR testing.

Generally, an attenuated immunogenic L. intracellularis strain is produced after continuous culture for between at least about 150 and 250 days, during which time the culture is passaged at least about 7 to about 12 times. Other attenuated cultures may be produced by varying these figures so long as the monitoring and selection methods taught herein are employed.

A vaccine is then prepared comprising an immunologically effective amount of the attenuated L. intracellularis in a pharmaceutically acceptable carrier. The combined immunogen and carrier may be an aqueous solution, emulsion or suspension. An immunologically effective amount is determinable by means known in the art without undue experimentation given the teachings contained herein. In general, the quantity of immunogen will be between 50 and 500 micrograms, and preferably between 10⁷ and 10⁹ TCID₅₀, when purified bacteria are used.

The L. intracellularis bacteria grown according to the method of the instant invention, or components derived from such bacteria, can be used as an antigen in an ELISA or other immunoassay, such as an immunofluorescent antibody test (“IFA”), to detect antibodies to L. intracellularis in the serum and other body fluids of animals suspected of being infected with the bacteria. The presently preferred immunoassay is an IFA as described in Example 1 of U.S. Pat. No. 5,885,823. Alternatively, the bacteria grown according to the invention can be used in a Western Blot assay.

WO 96/39629 and WO 05/011731 describe the cultivation of Lawsonia intracellularis, attenuated Lawsonia intracellularis and its administration.

In an advantageous embodiment, live Lawsonia intracellularis is modified-live Lawsonia intracellularis. In another advantageous embodiment, live Lawsonia intracellularis is attenuated Lawsonia intracellularis.

In an advantageous embodiment, the vaccine of the present invention has a dosage of Lawsonia intracellularis of about 10³ to 10⁹ bacteria/Kg of body weight, preferably of about 10⁵ to 10⁷ bacteria/Kg of body weight.

In an advantageous embodiment, the vaccine of the present invention has a dosage of the antigen of Lawsonia intracellularis of about 10⁵ to about 10⁷ of Lawsonia intracellularis bacteria.

In an advantageous embodiment, the antigen of Lawsonia intracellularis is lyophilized.

In an advantageous embodiment, the antigen of Lawsonia intracellularis in the vaccine of the present invention is the antigen included in Enterisol® Ileitis.

In an advantageous embodiment, the Lawsonia intracellularis vaccine is an Enterisol® Ileitis vaccine.

A preferred method of immunization or of vaccination consists in the administration of the vaccine according to the invention by systemic administration such as the intramuscular route.

In one aspect, the vaccine of the present invention may comprise an antigen of PCV. Accordingly, in one aspect of the present invention, an immunogenic composition for eliciting a protective immune response in a pig against PCV is provided. In the context of the present invention, plasmid constructs encoding and expressing PCV immunogens (antigens) may be used. Furthermore, methods of vaccination and DNA vaccines are described herein. In addition, the invention relates to methods of producing or of formulating these vaccines. Inactivated PCV vaccines (see, e.g., U.S. Pat. No. 6,517,843) are also contemplated.

PCV ORF1 and ORF2 according to Meehan 1998 encode proteins with predicted molecular weights of 37.7 kD and 27.8 kD, respectively. ORF3 and ORF4 (according to Meehan et al. 1998, correspond to ORF7 and ORF10 respectively in WO9918214) encode proteins with predicted molecular weights of 11.9 and 6.5 kD, respectively. The sequence of these ORFs is also available in Genbank AF055392. They can also be incorporated in plasmids and be used in accordance with the invention alone or in combination, e.g. with ORF1 and/or ORF2 and/or ORF3.

The other PCV ORFs 1-3 and 5, 6, 8-9, 11-12 disclosed in U.S. Pat. No. 6,391,314 (COLs 1-3 and 5, 6, 8-9, 11-12 in WO-A-9918214), may be used under the conditions described here, in combination or otherwise with each other or with the ORFs 1 and 2 as defined here.

This also encompasses the use of equivalent sequences in the leaning given above, in particular those ORFs coming from various PCV strains cited herein. The term “equivalent sequences” as used herein may refer to those sequences which come from a PCV strain having an ORF2 and/or an ORF 1 which have a homology or identity as defined further below with the corresponding ORF of strain Imp 1010. For ORF3 according to Meehan, it can also be said that homology or identity has to be for instance equal or greater than 80%, in particular than 85%, preferably than 90% or 95% with the ORF3 of the strain Imp1010. For ORF4 according to Meehan 1998, it can be equal or greater than 86%, in particular than 90%, preferably than 95% with ORF4 of strain Imp1010.

From the genomic nucleotide sequence, e.g. those disclosed in WO-A-99 18214, it is routine art to determine the ORFs using a standard software, such as MacVector™. Also, alignment of genomes with that of strain 1010 and comparison with strain 1010 ORFs allows the one skilled in the art to readily determine the ORFs on the genome for another strain (e.g. those disclosed in WO-A-99 18214). Using a software or making alignment is routine to the skilled person and may give directly access to equivalent ORFs.

The PCV3 ORF2 and the PCV3 genome sequences were derived from KT869077 (GenBank). Preferably, the PCV antigen of the vaccine of the present invention is a PCV1, PCV2 and/or PCV3 antigen. Preferably, the PCV antigen of the vaccine of the present invention is a recombinant polypeptide.

In a preferred aspect, the polypeptide of the present disclosure is a recombinant PCV1, PCV2 or PCV3 ORF2 protein, such as a recombinant baculovirus-expressed PCV3 ORF2 protein or, preferably, a recombinant baculovirus-expressed PCV2 ORF2 protein. The term “recombinant ORF2 protein”, as used herein, in particular refers to a protein molecule which is expressed from a recombinant DNA molecule, such as a polypeptide, which is produced by recombinant DNA techniques. An example of such techniques includes the case when DNA encoding the expressed protein is inserted into a suitable expression vector, preferably a baculovirus expression vector, which is in turn used to transfect, or in case of a baculovirus expression vector to infect, a host cell to produce the protein or polypeptide encoded by the DNA. The term “recombinant ORF2 protein”, as used herein, thus in particular refers to a protein molecule, which is expressed from a recombinant DNA molecule.

In other words, the PCV antigen of the vaccine of the present invention is preferably a recombinant polypeptide expressed (encoded) by a PCV ORF gene, preferably a PCV ORF2 gene, most preferably a PCV2 ORF2 gene.

The PCV antigen of the vaccine of the present invention is preferably a recombinant polypeptide expressed from (encoded by) a baculovirus cell.

The PCV antigen of the vaccine of the present invention is preferably a recombinant polypeptide expressed from (encoded by) a PCV ORF gene, preferably a PCV ORF2 gene, most preferably a PCV2 ORF2 gene and expressed in baculovirus cell.

The PCV antigen of the vaccine of the present invention is preferably the antigen of PCV included in Ingelvac CircoFLEX® or 3FLEX®.

According to a particular example, the recombinant PCV1, PCV2 or PCV3 ORF2 protein is produced by a method with the following steps: The gene for PCV1, PCV2 or PCV3 ORF2 is cloned into a baculovirus transfer vector; the transfer vector is used to prepare recombinant baculovirus containing said gene by homologous recombination in insect cells; and the PCV1, PCV2 or PCV3 ORF2 protein is then expressed in insect cells during infection with the recombinant baculovirus.

It is further understood that the term “recombinant PCV protein consisting of a sequence” in particular also concerns any cotranslational and/or posttranslational modification or modifications of the sequence affected by the cell in which the polypeptide is expressed. Thus, the term “recombinant PCV ORF2 protein consisting of a sequence”, as described herein, is also directed to the sequence having one or more modifications effected by the cell in which the polypeptide is expressed, in particular modifications of amino acid residues effected in the protein biosynthesis and/or protein processing, preferably selected from the group consisting of glycosylations, phosphorylations, and acetylations.

Preferably, the recombinant PCV1, PCV2 or PCV3 ORF2 protein according to the disclosure is produced or obtainable by a baculovirus expression system, in particular in cultured insect cells.

The word “plasmid” is here intended to cover any DNA transcription unit in the form of a polynucleotide sequence comprising the PCV sequence to be expressed and the elements necessary for its expression in vivo. The circular plasmid form, supercoiled or otherwise, is preferred. The linear form is also included within the scope of the invention.

In the context of the present invention, particularly the plasmids of U.S. Pat. No. 6,943,152 may be used. Each plasmid comprises a promoter capable of ensuring, in the host cells, the expression of the inserted gene under its control. It is in general a strong eukaryotic promoter and in particular a cytomegalovirus early promoter CMV-IE, of human or murine origin, or optionally of other origin such as rat or guinea pig. More generally, the promoter is either of viral origin or of cellular origin. As a viral promoter other than CMV-IE, there may be mentioned the SV40 virus early or late promoter or the Rous Sarcoma virus LTR promoter. It may also be a promoter from the virus from which the gene is derived, for example the promoter specific to the gene. As cellular promoter, there may be mentioned the promoter of a cytoskeleton gene, such as for example the desmin promoter, or alternatively the actin promoter. When several genes are present in the same plasmid, they may be provided in the same transcription unit or in two different units.

The plasmids may also comprise other transcription regulating elements such as, for example, stabilizing sequences of the intron type, preferably intron II of the rabbit beta-globin gene (van Ooyen et al. Science, 1979, 206: 337-344), signal sequence of the protein encoded by the tissue plasminogen activator gene (tPA; Montgomery et al. Cell. Mol. Biol. 1997, 43: 285-292), and the polyadenylation signal (polyA), in particular of the bovine growth hormone (bGH) gene (US-A-5,122,458) or of the rabbit beta-globin gene.

“Sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A sequence comparison may be carried out over the entire lengths of the two sequences being compared or over fragments of the two sequences. Sequence identity may be carried out over a region, for example, twenty, fifty, one hundred or more contiguous amino acid residues, however, typically, the comparison will be carried out over the full length of the two sequences being compared.

A “conservative substitution” refers to the substitution of an amino acid residue or nucleotide with another amino acid residue or nucleotide having similar characteristics or properties including size, hydrophobicity, etc., such that the overall functionality does not change significantly.

In the context of the present invention also PCV1 or PCV2 or PCV3 proteins with mutations may be used, such as but not limited to mutations of the capsid protein. Despite the divergence of the capsid amino acid sequences between PCV2 and beak and feather disease virus (BFDV), the crystal structures are very similar despite their sequence divergence. Advantageously, the mutations of PCV3 are to stabilize virus-like particles (VLPs). The PCV3 capsid protein should self-assemble into a VLP, however, the level of expression of the PCV3 protein is significantly lower as compared to the PCV2 capsid protein. Specifically, only about 20% of the protein assembles into VLPs whereas the remaining 80% of the protein aggregates into an insoluble fraction. The mutations of the PCV3 capsid protein disclosed in International Patent Application Serial No. PCT/US2020/026930 may be used in the context of the present invention.

Assays and techniques suitable for use in the context of the present invention include those that have been used for the tracking or quantifying the assembly and disassembly of porcine circovirus capsid (ORF2) protein into virus-like particles (VLPs) and these include: enzyme-linked immunosorbent assay (ELISA), SDS/PAGE optionally with silver stain or coomassie stain, western blot or immunoblot, size exclusion chromatography (SEC), dynamic light scattering (DLS) or multi-angled light scattering (MALS), transmission electron microscopy (TEM), analytical ultracentrifugation, and fluorescence spectroscopic analysis (FSA) optionally coupled with high performance liquid chromatography (HPLC). Additional suitable techniques may also include: agarose gel retardation tests of protein-nucleic acid complexes, immune diffusion tests e.g. single radial immunodiffusion (SRID), nanoparticle tracking analysis (NTA), metabolic labelling and chemiluminescent enzyme-based assays. Each of these assays is well-known in the art and is described in, for example, Fang, Mingli et al. “Detection of the Assembly and Disassembly of PCV2b Virus-Like Particles Using Fluorescence Spectroscopy Analysis” Intervirology vol. 58, 2015, pp. 318-323; Thompson, Christine et al. “Analytical technologies for influenza virus-like particle candidate vaccines: challenges and emerging approaches” Virology Journal vol 10, 2013, p. 141; Steppert, Petra et al. “Quantification and characterization of virus-like particles by size-exclusion chromatography and nanoparticle tracking analysis” Journal of Chromatography A vol. 1487, 2017, pp. 89-99; Yadav, Shalini et al. “A facile quantitative assay for viral particle genesis reveals cooperativity in virion assembly and saturation of an antiviral protein” Virology, vol 429, No. 2, 2012, pp. 155-162; and Zeltins, Andris “Construction and Characterization of Virus-Like Particles: A Review” Molecular Biotechnology vol. 53, 2013, pp. 92-107, each of which is incorporated herein by reference in its entirety.

The development of a recombinant baculovirus containing the PCV3 ORF2 gene under control of the baculovirus polyhedrin promoter (BaculoG/PCV3 ORF2 Clone 4B4-2E12 Pre-MSV p8; lot no. 3624-039) is described in Example 1 of International Patent Application Serial No. PCT/US2020/026930. In some embodiments, the use of such a recombinant baculovirus-expressed protein described in said Example 1 in a vaccine may encompass killed and/or inactivated versions of the recombinant virus. Alternatively, in some vaccines, a recombinant virus, for example similar to that shown in Example 1 of International Patent Application Serial No. PCT/US2020/026930, may be used as a live, modified virus.

In some embodiments, the amplified PCV ORF2 coding sequence may be subcloned into a baculovirus transfer vector utilizing the flanking restriction sites to generate the desired transfer vector. For example, the amplified PCV ORF2 coding sequence may be subcloned into a baculovirus transfer vector utilizing the flanking restriction sites to generate transfer vectors. Recombinant baculovirus may be generated by co-transfection of insect cells with a transfer vector and baculovirus DNA. Baculovirus DNA used may include linearized and/or circular baculovirus DNA. For example, in an embodiment, recombinant baculovirus may be generated by co-transfection of Sf9 (Spodoptera frugiperda) insect cells with a transfer vector and linearized BaculoGold™ baculovirus DNA. The linearized baculovirus DNA may be derived from Autographa californica nuclear polyhedrosis virus (AcNPV) and may contain a lethal deletion in the polyhedrin locus, therefore, rescue of viable baculovirus may be generated upon co-transfection with a transfer vector. The resulting recombinant baculovirus may include a PCV ORF2 coding sequence under control of the baculovirus polyhedrin promoter. The recombinant baculovirus may be amplified on Sf9 insect cells and subsequently purified by limiting dilution cloning on Sf9 insect cells. In some embodiments, a full length circular baculovirus DNA such as Bac-to-Bac may be used. For example, Bac-to-Bac may use transposon-mediated recombination to insert a gene of interest into a polyhedron locus. Other methods known in the art may also be used. In some embodiments, a method may be chosen based on the potential stability of the method during commercialization. For example, baculoviruses that confer increased stability in the vaccine may be selected.

In some embodiments, after seeding flasks with of a master cell culture, the flasks may be incubated at a predetermined temperature and for a specific time frame. For example, a culture may be incubated at 27° C. for four hours. Each flask may then be seeded with a recombinant baculovirus containing the PCV ORF2 gene. For example, a plasmid containing an ORF2 gene can be co-transfected with BaculoGold® (BD Biosciences Pharmingen) baculovirus DNA into Sf+ insect cells (Protein Sciences, Meriden, CT) to generate a recombinant baculovirus containing a ORF2 gene. The recombinant baculovirus containing the ORF2 gene may be plaque-purified and Master Seed Virus (MSV) propagated on the SF+ cell line, aliquotted, and stored at -70° C. The MSV may be positively identified as ORF2 baculovirus by PCR-RFLP using baculovirus specific primers. Insect cells infected with ORF2 baculovirus to generate MSV or Working Seed Virus may express ORF2 antigen as detected by polyclonal serum or monoclonal antibodies in an indirect fluorescent antibody assay. Additionally, the identity of the ORF2 baculovirus may be confirmed by N-terminal amino acid sequencing. The ORF2 baculovirus MSV may also be tested for purity in accordance with 9 C.F.R. Sections 113.27 (c), 113.28, and 113.55. Each recombinant baculovirus seeded into the spinner flasks may have varying multiplicities of infection (MOIs).

After being seeded with the baculovirus, the flasks may be incubated at 27 ± 2° C. for 7 days and may also be agitated at 100 rpm during that time. The flasks may use ventilated caps to allow for air flow. Samples from each flask may be taken every 24 hours for the next 7 days. After extraction, each sample may be centrifuged, and both the pellet and the supernatant are separated and then microfiltered through a 0.45-1.0 µm pore size membrane.

The amount of ORF2 in the resulting samples may then be quantified via an ELISA assay. The ELISA assay may be conducted with an anti-PCV antibody diluted to 1:6000 in 0.05 M Carbonate buffer (pH 9.6). 100 µL of the antibody may then be placed in the wells of the microtiter plate, sealed, and incubated overnight at 37° C. The plate is then washed three times with a wash solution which comprised 0.5 mL of Tween 20 (Sigma, St. Louis, MO), 100 mL of 1OX D-PBS (Gibco Invitrogen, Carlsbad, CA) and 899.5 mL of distilled water. Subsequently, 250 µL of a blocking solution (5 g Carnation Non-fat dry milk (Nestle, Glendale, CA) in 10 mL of D-PBS QS to 100 mL with distilled water) is added to each of the wells. The next step is to wash the test plate and then add pre-diluted antigen. The pre-diluted antigen is produced by adding 200 µL of diluent solution (0.5 mL Tween 20 in 999.5 mL D-PBS) to each of the wells on a dilution plate. The sample is then diluted at a 1:240 ratio and a 1:480 ratio, and 100 µL of each of these diluted samples is then added to one of the top wells on the dilution plate (i.e. one top well received 100 µL of the 1:240 dilution and the other received 100 µL of the 1:480 dilution). Serial dilutions may then be done for the remainder of the plate by removing 100 µL from each successive well and transferring it to the next well on the plate. Each well is mixed prior to doing the next transfer. The test plate washing includes washing the plate three times with the wash buffer. The plate is then sealed and incubated for an hour at 37° C. before being washed three more times with the wash buffer. The detection antibody used is an antibody to PCV ORF2. It is diluted to 1 to 300 in diluent solution, and 100 µL of the diluted detection antibody was then added to the wells. The plate is then sealed and incubated for an hour at 37° C. before being washed three times with the wash buffer. Conjugate diluent is then prepared by adding normal rabbit serum (Jackson Immunoresearch, West Grove, PA) to the diluent solution to 1% concentration.

Conjugate antibody Goat anti-mouse (H+1)-HRP (Jackson Immunoresearch) is diluted in the conjugate diluent to 1:10,000. 100 µL of the diluted conjugate antibody is then added to each of the wells. The plate is then sealed and incubated for 45 minutes at 37° C. before being washed three times with the wash buffer. 100 µL of substrate (TMB Peroxidase Substrate, Kirkgaard and Perry Laboratories (KPL), Gaithersburg, MD), mixed with an equal volume of Peroxidase Substrate B (KPL) is added to each of the wells. The plate is incubated at room temperature for 15 minutes. 100 µL of IN HCL solution is then added to all of the wells to stop the reaction. The plate is then run through an ELISA reader.

Advantageously insect cells can be cultured, and the PCV ORF2 protein produced, under serum-free conditions; such as the serum-free insect cells of USP 6,103,526 (expresSF+ cell line). Other insect cell lines include, but are not limited to, Spodoptera frugiperda (Sf) cell lines, such as Sf21, Sf9, and expresSF+1 (SF+), BTI-TN5B1 (High Five) cells from the Trichoplusia ni cabbage looper, and BmN cells from the Bombyx mori silkworm are widely being used in baculovirus research and for recombinant protein production.

The adjuvants, cell culture supernatants, preservatives, stabilizing agents, viral vectors, immunomodulatory agents and dosages disclosed in U.S. Pat. Nos. 9610345 and 9669087 are contemplated, both incorporated herein by reference.

In the context of the present invention, immunogenic preparations and DNA vaccines may also be used comprising at least one plasmid disclosed herein, encoding and expressing one of the PCV1 or PCV2 or PCV3 immunogens, preferably one of the above-mentioned ORFs, in addition a veterinarily acceptable vehicle or diluent, with optionally, in addition, a veterinarily acceptable adjuvant. In one embodiment, the adjuvant may include CARBOPOL™ or ImpranFLEX®.

In an embodiment, an immunogenic composition may refer to a composition that includes in a one ml dose i) at least some PCV ORF2 protein, ii) baculovirus expressing said PCV ORF2 protein, iii) cell culture, iv) an inactivating agent (e.g, BEI) having a concentration in a range from about 2 to about 8 mM, v) a neutralization agent (e.g., sodium thiosulfate) in equivalent amounts to the inactivating agent; and vi) a predetermined amount of adjuvant (e.g., CARBOPOL® 971 or ImpranFLEX®.), and vii) phosphate salt in a physiologically acceptable concentration.

Most preferably, the composition provided herewith, contains PCV ORF2 protein recovered from the supernatant of in vitro cultured cells, wherein said cells were infected with a recombinant viral vector containing PCV ORF2 DNA and expressed PCV ORF2 protein, and wherein said cell culture was treated with about 2 to about 8 mM BEI, preferably with about 5 mM BEI to inactivate the viral vector, and an equivalent concentration of a neutralization agent, preferably sodium thiosulfate solution in a final concentration of about 2 to about 8 mM, preferably of about 5 mM. The quantity of PCV antigen-encoding DNA used in the vaccine according to the present invention is between about 10 µg and about 2000 µg, and preferably between about 50 µg and about 1000 µg. Persons skilled in the art will have the competence necessary to precisely define the effective dose of DNA to be used for each immunization or vaccination protocol.

The dose volumes may be between 0.5 and 5 ml, preferably between 2 and 3 ml.

In another embodiment, the present invention encompasses a method for eliciting an immune response or an immunological response or a protective immune or immunological response inter alia against porcine circovirus (PCV) comprising parenterally or subcutaneously administering to a porcine of a single shot, single administration or single dose (i) at least 2 µg to about 400 µg of a PCV ORF2 recombinant protein expressed by a baculovirus system and (ii) a veterinary-acceptable carrier comprising a solvent, a dispersion media, a coating, a stabilizing agent, a diluent, a preservative, an antimicrobial agent, an antifungal agent, an isotonic agent, an adsorption delaying agent, an adjuvant, cell culture supernatant, a stabilizing agent, a viral or expression vector, an immunomodulatory agent and/or any combination thereof.

In an advantageous embodiment, the PCV vaccine is Ingelvac CircoFLEX® (see, e.g., WO 2006/072065).

WO2006/072065 and WO2008/076915 describe the generation of the PCV vaccine, its formulation and its administration.

In an advantageous embodiment, the vaccine has a dosage of about 2 µg to about 400 µg of the antigen of PCV. Thus, the vaccine has a dosage of about 2 µg to about 400 µg of the PCV2 ORF2 protein.

In another advantageous embodiment, the vaccine has a dosage of about 4 µg to about 200 µg of the antigen of PCV. Thus, the vaccine has a dosage of about 4 µg to about 200 µg of the PCV2 ORF2 protein.

In yet another advantageous embodiment, the vaccine has a dosage of about 10 µg to about 100 µg of the antigen of PCV. Thus, the vaccine has a dosage of about 10 µg to about 100 µg of the PCV2 ORF2 protein.

In an advantageous embodiment, the vaccine according to the invention comprises an antigen of Lawsonia intracellularis and one or more antigens of PCV, wherein the antigen of Lawsonia intracellularis is live Lawsonia intracellularis, preferably attenuated Lawsonia intracellularis or modified-live Lawsonia intracellularis.

In an advantageous embodiment, the vaccine according to the invention comprises an antigen of Lawsonia intracellularis and the antigen of PCV included in Ingelvac CircoFLEX® or 3FLEX®, wherein the antigen of Lawsonia intracellularis is live Lawsonia intracellularis, preferably attenuated Lawsonia intracellularis or modified-live Lawsonia intracellularis.

In an advantageous embodiment, the vaccine according to the invention has a dosage of the antigen of Lawsonia intracellularis of about 10³ to 10⁹ bacteria/Kg of body weight, preferably of about 10⁵ to 10⁷ bacteria/Kg of body weight and comprises the antigen of PCV included in Ingelvac CircoFLEX® or 3FLEX®, wherein the antigen of Lawsonia intracellularis is live Lawsonia intracellularis, preferably attenuated Lawsonia intracellularis or modified-live Lawsonia intracellularis.

In an advantageous embodiment, the vaccine according to the invention has a dosage of the antigen of Lawsonia intracellularis of about 10⁵ to about 10⁷ of Lawsonia intracellularis bacteria and comprises the antigen of PCV included in Ingelvac CircoFLEX® or 3FLEX®, wherein the antigen of Lawsonia intracellularis is live Lawsonia intracellularis, preferably attenuated Lawsonia intracellularis or modified-live Lawsonia intracellularis.

In an advantageous embodiment, the vaccine according to the invention comprises the antigen of Lawsonia intracellularis included in Enterisol® Ileitis and one or more antigens of PCV.

In an advantageous embodiment, the vaccine according to the invention comprises the antigen of Lawsonia intracellularis included in Enterisol® Ileitis and one or more antigens of PCV, wherein PCV is PCV1, PCV2 or PCV3.

In an advantageous embodiment, the vaccine according to the invention comprises the antigen of Lawsonia intracellularis included in Enterisol® Ileitis and one or more antigens of PCV, wherein the antigen(s) of PCV is/are (a) recombinant polypeptide(s), preferably expressed in a baculovirus cell.

In an advantageous embodiment, the vaccine according to the invention comprises the antigen of Lawsonia intracellularis included in Enterisol® Ileitis and one or more antigens of PCV, wherein the antigen(s) of PCV is/are (a) recombinant polypeptide(s) expressed by a PCV ORF gene, preferably expressed in a baculovirus cell.

In an advantageous embodiment, the vaccine according to the invention comprises the antigen of Lawsonia intracellularis included in Enterisol® Ileitis and one or more antigens of PCV, wherein the antigen(s) of PCV is/are (a) recombinant polypeptide(s) expressed by a PCV ORF2 gene, preferably expressed in a baculovirus cell.

In a very advantageous embodiment, the vaccine according to the invention comprises the antigen of Lawsonia intracellularis included in Enterisol® Ileitis and the antigen of PCV included in Ingelvac CircoFLEX® or 3FLEX®.

A preferred method of immunization or of vaccination consists in the systemic administration of the vaccine according to the invention and as described directly above. Systemic administration methods are described herein and include but are not limited to intramuscular and intradermal administration.

Accordingly, a preferred method of immunization or of vaccination consists in the administration of the vaccine according to the invention by the intramuscular route.

In one aspect, the vaccine of the present invention may comprise an antigen of M. hyo. Accordingly, in one aspect of the present invention, an immunogenic composition for eliciting a protective immune response in a pig against M. hyo. is provided. Even more preferably, the amount of the M. hyo. antigen in each dose has a relative potency (RP) value of at least 1.22, wherein a relative potency value of 1.22 means that at least 95% and preferably 100% of mice receiving an administration of one-fortieth (1/40) of such amount of M. hyo. antigen develop a detectable amount of antibodies within or at 21 days post treatment in a M. hyo. specific antibody detection assay. Thus, the 40-fold amount of M. hyo. antigen that is needed to induce a detectable M. hyo. specific antibody response in at least 95% and preferably 100% of mice within or at 21 days post treatment is sufficient to confer a protective immune response against, reduces the incidence of, and/or lessens the severity of or prevents the clinical signs associated with a M. hyo. infection. In other words, the amount of M. hyo. antigen as described above has been shown to be able to overcome any negative interference with a PCV antigen, when mixed and administered as a combination vaccine. In some preferred forms, the composition further includes or comprises an adjuvant. A variety of adjuvants will be useful in connection with the present invention and can be selected by those of skill in the art, but carbomer and even more preferably CARBOPOL® (high molecular weight crosslinked polyacrylic acid polymer) or ImpranFLEX® are particularly preferred. Advantageously, the immunogenic composition of the present invention confers a protective immune response against, reduces the incidence of, and/or lessens the severity of and/or prevents the clinical signs associated with a M. hyo. Infection, preferably when administered to a pig as a single dose administration. Such a single dose elicits a duration of immunity of at least 100, more preferably at least 110, even more preferably at least 120, still more preferably at least 130, even more preferably at least 140, still more preferably at least 150, even more preferably at least 160, still more preferably at least 170, even more preferably at least 180, and most preferably at least 184 days when administered to a pig. In other words, one dose of the immunogenic composition of the present invention, without boosters or subsequent doses, provides an animal or group of animals with a reduced incidence of or lessened severity of clinical signs of infection from M. hyo. for at least 100 (110, 120, 130, 140, 150, 160, 170, 180, etc) and most preferably at least 184 days. With respect to the antibody detection assay, those of skill in the art will be able to identify and utilize appropriate products. ELISA assays and especially the IDEXX Herdchek M. hyo. Test Kit™ (IDEXX Laboratories, Inc., Westbrook, Me.) are preferred. In particular, the IDEXX Herdchek M. hyo. Test Kit™ (IDEXX Laboratories, Inc., Westbrook, Me.) may be used as a reference assay in the context of the present invention.

As used herein, a “protective immune response” refers to a reduced incidence of or reduced severity of clinical, pathological, or histopathological signs of M. hyo. infection up to and including the complete prevention of such signs. A “protective immune response” may be triggered by an immunologically effective amount of the antigen or vaccine.

The term “M. hyo. antigen” refers to any composition of matter that comprises at least one antigen that can induce, stimulate or enhance the immune response against M. hyo. infection, when administered to an animal, preferably a pig. Preferably, said M. hyo. antigen is a whole M. hyo. bacterin, preferably in an inactivated form, a live modified or attenuated M. hyo. bacterium, a chimeric virus that comprises at least an immunogenic amino acid sequence of M. hyo., or any other polypeptide or component that comprises at least an immunogenic amino acid sequence of M. hyo. Preferably, the M. hyo. antigen is an inactivated M. hyo. bacterin. More preferably, the M. hyo. antigen is derived from the M. hyo. J-strain. Most preferably, the M. hyo. bacterin is the inactivated M. hyo. bacterin that is included in INGELVAC® MYCOFLEX vaccine (Boehringer Ingelheim Vetmedica Inc, St Joseph, Mo., USA) or is INGELVAC® MYCOFLEX. However, the M. hyo. antigen that can be used according to the invention can also be selected from any one which is included in the following vaccine compositions: PORCILIS M. HYO, MYCO SILENCER® BPM, MYCO SILENCER® BPME, MYCO SILENCER® ME, MYCO SILENCER® M, MYCO SILENCER® ONCE, MYCO SILENCER® MEH (all of Intervet Inc., Millsboro, Del., USA) STELLAMUNE MYCOPLASMA™ (Pfizer Inc., New York, N.Y., USA), SUVAXYN MYCOPLASMA™, SUVAXYN M. HYO™, SUVAXYN MH-ONE™ (all of Fort Dodge Animal Health, Overland Park, Kans., USA (Wyeth). Advantageously, a dose of 2 ml of M. hyo. supernatant and/or bacterin is contemplated.

Available M. hyo. vaccines are made from killed whole cell mycoplasma preparations (bacterins). Accordingly, “bacterin” as used herein refers to whole cell preparations of bacteria, specifically of M. hyo, which are preferably killed whole cell preparations. When the vaccine or antigen is described herein to be a “supernatant”, said supernatant may be the soluble fraction/portion of a (killed) whole cell preparation. The present invention also contemplates the use of a soluble portion of a M. hyo. whole cell preparation, wherein the soluble portion of the M. hyo. preparation is substantially free of both (i) IgG and (ii) immunocomplexes comprised of antigen bound to immunoglobulin (see, e.g., U.S. Pat. No. 10,206,991). In some embodiments, the soluble portion of the M. hyo. preparation includes at least one M. hyo. protein antigen. In other embodiments, the soluble portion of the M. hyo. preparation includes two or more M. hyo. protein antigens. In one embodiment, the M. hyo. supernatant fraction includes one or more of the following M. hyo. specific protein antigens: M. hyo. proteins of approximately 46 kD (p46), 64 kD (p64) and 97 kD (p97) molecular weights. In another embodiment, the supernatant fraction at least includes the p46, p64 and p97 M. hyo. protein antigens. The M. hyo. protein of approximately 64 kD (p64) may be alternatively referred to herein as the p65 surface antigen from M. hyo. described by Kim et al. (Infect. Immun. 58(8):2637-2643 (1990)), as well as in U.S. Pat. No. 5,788,962.

Any M. hyo. strain may be used as a starting material to produce the soluble portion of the M. hyo. preparation. Suitable strains of M. hyo. may be obtained from commercial or academic sources, including depositories such as the American Type Culture Collection (ATCC) (Manassas, Va.) and the NRRL Culture Collection (Agricultural Research Service, U.S. Department of Agriculture, Peoria, Ill.). The ATCC alone lists the following six strains of M. hyo. for sale: M. hyo. ATCC 25095, M. hyo. ATCC 25617, M. hyo. ATCC 25934, M. hyo. ATCC 27714, M. hyo. ATCC 27715, and M. hyo. ATCC 25934D. A preferred strain of M. hyo. for use in the embodiments of this invention is identified as strain P-5722-3, ATCC #55052, deposited on May 30, 1990 pursuant to the accessibility rules required by the U.S. Patent and Trademark Office. In view of the widespread dissemination of the disease, strains may also be obtained by recovering M. hyo. from lung secretions or tissue from swine infected with known strains causing mycoplasmal pneumonia in swine.

Injection timing is flexible. Compositions as described herein can be used as early as three weeks of age through the time when pigs leave the nursery with the objective of vaccinating at least 2 weeks prior to exposure to M. hyo. The vaccine according to the invention may be applied in any conventional manner including intradermally, intratracheally, or intravaginally. The vaccine according to the invention may also be applied by systemic administration. The composition preferably may be applied intramuscularly or intradermally.

WO2009/126356, US8444989, US 8852613 and US 8940309 describe the generation of M. hyo bacterins, its formulation and its administration.

In an advantageous embodiment, the amount of the M. hyo. antigen in each dose has a relative potency (RP) value of at least 1.22,

In another advantageous embodiment, the M. hyo. bacterin has an antigen amount between 5 log₁₀ and 8 log₁₀ per ml before the inactivation.

In an advantageous embodiment, the M. hyo. vaccine is Ingelvac MycoFLEX®. Accordingly, in an advantageous embodiment, the antigen of M. hyo. is the antigen of M. hyo. included in Ingelvac MycoFLEX®.

A preferred method of immunization or of vaccination consists in the administration of the vaccine according to the invention by systemic administration such as the intramuscular route.

In one aspect, the vaccine of the present invention comprises an antigen of PRRSV. Accordingly, in one aspect of the present invention, an immunogenic composition for eliciting a protective immune response in a pig against PRRSV is provided. The viral envelope proteins of PRRSV are generally categorized into major and minor proteins based on abundance of proteins in the virion. The major viral envelope proteins are gp5 (ORF 5) and M (ORF 6) and form a dimer. The minor envelope proteins are gp2 (ORF2), gp3 (ORF3), gp4 (ORF4) and E (ORF2b) and probably a newly identified viral protein gp5a (ORF 5a). The active antigenic component can include the ORF4, ORF5, ORF6, or ORF7 from PRRSV virus.

The recombinant PRRSV antigen may be expressed in a vectored PRRSV vaccine or composition that comprises one or more engineered, recombinant adenovirus vectors that harbor and express certain PRRSV antigens, and optionally a pharmaceutically or veterinarily acceptable carrier, adjuvant, excipient, or vehicle. Advantageous, the vector is an adenovirus vector although other vectors, such as a baculovirus, are also contemplated.

The PRRSV may be any strain, as the novel and inventive compositions and methods disclosed herein are universally applicable to all known and yet to be discovered PRRSV strains. PRRSV virus exists as two genotypes referred to as “US” and “EU” type which share about 50% sequence homology (Dea S et al. (2000). Arch Virol 145:659-88). These two genotypes can also be distinguished by their immunological properties. Most sequencing information on various isolates is based on the structural proteins, namely the envelope protein GP5 which accounts for only about 4% of the viral genome, while only little is known on the non-structural proteins (nsp). Isolation of PRRSV and manufacture of vaccines have been described in a number of publications (WO 92/21375, WO 93/06211, WO 93/03760, WO 93/07898, WO 96/36356, EP 0 676 467, EP 0 732 340, EP 0 835 930, US 10,039,821). The PRRSV antigen includes PRRSV minor proteins (e.g. gp2, gp3, gp4, gp5a, gp5 or E), in any combination, and optionally includes additional PRRSV major proteins (e.g. gp5 or M). For example, the PRRSV antigens could be displayed on the surface of virus-like particles (VLPs). In other embodiments, soluble versions of the antigens could be administered to the host animal, wherein oligomerization (including trimerization) of the proteins with each other, or additionally, with components of VSV-G, or other viral proteins or any oligomerization (including trimerization motifs) (e.g. motifs from bacterial GCN4, and the like). Moreover, the TM/CT domains of Type I viral surface glycoproteins are envisioned to accomplish the same purpose as, and are therefore interchangeable with, the corresponding domains from VSV-G.

In some embodiments, the PRRSV vaccine is a recombinant vaccine. In this instance, one or more vectors comprise either: a nucleotide sequence encoding a PRRSV E antigen, polypeptide, ectodomain or variant thereof; or, a nucleotide sequence encoding a modified PRRSV gp2, gp3, gp4, gp5a, gp5 or M antigen, polypeptide, ectodomain, or variant thereof, wherein an existing cellular localization sequence of gp2, gp3, gp4, gp5a, gp5 or M has been replaced with a cell-surface expression determinant sequence from an heterologous gene. In some embodiments, the one or more vectors comprise a mixture of two vectors, a first vector expressing retargeted PRRSV minor proteins, and a second vector expressing re-targeted PRRSV major proteins.

In the context of the present invention, methods may be used for the production of live porcine reproductive and respiratory syndrome virus (PRRSV) for use in the production of vaccines and other compositions. In typical production methods, the virus is grown on a cell line that is permissive to PRRSV infection. However, in such general methods the cell line is grown to at or near confluence prior to infection with the PRRSV.

In an advantageous method, the cell line does not need to be planted and grown prior to infection with PRRSV, but rather that the PRRSV and the cell line may be added to the cell culture process concurrently. Said method, thus, provides the significant advantage of savings in time, cost and materials when the virus is being mass produced at commercial scale. The term commercial scale refers to volumes of cell culture in excess of 10 L. For example, commercial scale refers to a range of from 10 L to 3000 L production scale for live PRRSV. In more specific embodiments, the volume is from 30 L to about 300 L.

The herein described methods may be used for the production of any PRRSV strain, including but not limited to PRRSV strain deposited as ATCC VR 2332, VR 2385, VR 2386, VR 2429, VR 2474, and VR 2402; CNCM 1-1102, CNCM 1-1140, CNCM 1-1387, CNCM 1-1388, or ECACC V93070108. In particularly preferred embodiments, the methods of the invention are used to produce PRRSV strain 94881 deposited with the European Collection of Cell Cultures (ECACC) under the Accession Numbers ECACC 11012501 (parental strain) and ECACC 11012502 (high passage attenuated MSV) each deposited on Jan. 25, 2011 in accordance with the provisions of the Budapest Treaty, or any descendant or progeny of one of the aforementioned strains. The viruses grown may be any of the aforementioned viruses in their attenuated format. Alternatively, the viruses may be genetically modified to comprise one or more heterologous nucleic acids that encode further antigenic determinants of one or more swine diseases.

The skilled person will understand that there are a number of cell lines that are permissive to infection by PRRSV. Exemplary cells are cells porcine alveolar macrophage cells such as those derived from MARC-145 cells. Other cells that can be infected with the PRRSV include MA-104 cells; Baby Hamster Kidney (BHK) cells; Chinese Hamster Ovary (CHO) cells; and African Green Monkey kidney cells other than MA-104 cells or MARC-145 cells, such as VERO cells; that are transfected. In addition, the cells may be primary cells from a swine animal that have been adapted for long term growth in culture. Particularly suitable host cells are the simian cell line MA-104, Vero cells, or porcine alveolar macrophages. PRRSV preferentially grows in alveolar lung macrophages (Wensvoort et al., 1991). A few cell lines, such as CL2621 and other cell lines cloned from the monkey kidney cell line MA-104 (Benfield et al., 1992; Collins et al., 1992; Kim et al., 1993) are also susceptible to the virus and may be used in the large-scale production methods described herein.

In the exemplary method shown in Example 1 of U.S. Pat. No. 9,944,902, there is provided a concurrent process for the production of PRRSV 94881 MLV. While this procedure is shown for PRRSV 94881 MLV, the skilled person will understand that this procedure may be readily used for any PRRSV for which large scale production is required.

The viruses produced by the described production method may be used for providing a PRRSV antigen for use in the vaccine of the invention, particularly a MLV PRRSV.

The virus strains grown according to said methods may be virulent PRRS viruses, attenuated PRRS viruses or indeed PRRS viruses that have been modified to impart further desirable properties to them. This may be achieved by classical propagation and selection techniques, like continued propagation in suitable host cells to extend the attenuated phenotype. Alternatively, the strains may be genetically modified by directed mutation of the nucleic acid sequence of the genome of these strains by suitable genetic engineering techniques. The genome of PRRSV was completely or partly sequenced (Conzelmann et al., 1993; Meulenberg et al., 1993a, Murthaugh et al, 1995) and encodes, besides the RNA-dependent RNA polymerase (ORFs 1a and 1b), six structural proteins of which four envelope glycoproteins named GP2 (ORF2), GP3 (ORF3), GP4 (ORF4) and GP5 (ORF5), a non-glycosylated membrane protein M (ORF6) and the nucleocapsid protein N(ORF7) (Meulenberg et al. 1995, 1996; van Nieuwstadt et al., 1996). Immunological characterization and nucleotide sequencing of European and US strains of PRRSV has identified minor antigenic differences within strains of PRRSV located in the structural viral proteins (Nelson et al., 1993; Wensvoort et al., 1992; Murtaugh et al., 1995).

Indeed, an exemplary virus is PRRSV 94881 virus. While an attenuated strain is grown using the methods described herein, the virus may easily be a PRRSV 94881 virus that is made into a chimeric virus wherein the backbone of the PRRSV 94881 virus under ECACC Accession No. 11012502 or indeed the parent strain deposited under ECACC Accession No 11012501 is modified to replace the endogenous sequence of one or more of ORF 1a, ORF 1b, ORF 2, ORF 3, ORF 4, ORF 5, ORF 6, or ORF 7 with the corresponding ORF from a different strain of PRRS virus. For example, the different strain of the PRRS virus may be a different European strain such as Lelystad virus strain (Lelystad Agent (CDI-NL-2.91), or other strains such as those deposited under the Accession Numbers ECACC 04102703, ECACC 04102702, ECACC 04102704, CNCM Accession No. 1-1140, CNCM Accession No 1-1387, CNCM Accession No 1-1388, ATCC VR 2332, VR 2385, VR 2386, VR 2429, VR 2474, and VR 2402; CNCM I-1102, CNCM I-1140, CNCM I-1387, CNCM I-1388, or ECACC V93070108 or indeed may be a U.S. strain such as North American PRRS virus, pT7P129A; ATCC deposit VR-2332, ATCC deposit VR-2368; ATCC VR-2495; ATCC VR 2385, ATCC VR 2386, ATCC VR 2429, ATCC VR 2474, and ATCC VR 2402.

Recombinant techniques for preparing modified sequences are well known to those of skill in the art and usually employ construction of a full-length complementary DNA copy (infectious clones) of the viral genome which may then be modified by DNA recombination and manipulation methods (like site-directed mutagenesis etc.). This way, for example antigenic sites or enzymatic properties of viral proteins may be modified. Infectious clones of PRRS virus strains of European and North American genotype have been reported in the literature and may be grown using the methods of the invention.

Preferably, vaccines according to the present invention comprise modified live PRRSV comprising one or more of these strains alive in a suitable carrier, but inactivated virus may also be used to prepare killed vaccine (KV). MLV are typically formulated to allow administration of 10¹ to 10⁷ viral particles per dose, preferably 10³ to 10⁵ particles per dose, more preferably 10⁴ to 10⁵ particles per dose (4.0-5.0 log₁₀ TCID₅₀). The vaccine of the present invention may have a dosage of the antigen of PRRSV of about 10⁴ to about 10⁷ viral particles per dose. KV may be formulated based on a pre-inactivation titre of 10³ to 10¹⁰ viral particles per dose. The vaccine may comprise a pharmaceutically acceptable carrier, for example a physiological salt-solution. The vaccine may or may not comprise an adjuvant. An example of a suitable adjuvant is alpha-tocopherol acetate which can be obtained under the trade name Diluvac Forte®. Alternatively, for example aluminium-based adjuvants may be used.

Pigs can be infected by PRRSV via the oronasal route. Virus in the lungs is taken up by lung alveolar macrophages and in these cells replication of PRRSV is completed within 9 hours. PRRSV travels from the lungs to the lung lymph nodes within 12 hours and to peripheral lymph nodes, bone marrow and spleen within 3 days. At these sites, only a few cells stain positive for viral antigen. The virus is present in the blood during at least 21 days and often much longer. After 7 days, antibodies to PRRSV are found in the blood. The combined presence of virus and antibody in PRRS infected pigs shows that the virus infection can persist for a long time, albeit at a low level, despite the presence of antibody. During at least 7 weeks, the population of alveolar cells in the lungs is different from normal SPF lungs.

A vaccine may be presented in form of a freeze-dried preparation of the live virus, to be reconstituted with a solvent, to result in a solution for injection. Thus, after the harvesting steps of the described methods, the virus may be combined and freeze dried. The solvent may e.g. be water, physiological saline, or buffer, or an adjuvanting solvent. The solvent may contain adjuvants, for example alpha-tocopherol acetate. The reconstituted vaccine may then be injected into a pig, for example as an intramuscular or intradermal injection into the neck. For intramuscular injection, a volume of 2 ml may be applied, for an intradermal injection it is typically 0.2 ml. In a further aspect, the present invention therefore relates to a vaccine product, comprising in separate containers a freeze-dried composition of the virus, and a solvent for reconstitution, and optionally further containing a leaflet or label comprising instructions of use.

A vaccine prepared from a virus produced by a method described above may not only comprise one or more of the aforementioned strains, but may include further components active against PRRS or other porcine viral or bacterial diseases, such as Lawsonia intracellularis, PCV and/or M. hyo.. Therefore, the invention further relates to a vaccine as described, characterized in that it contains at least one further antigen active against a porcine disease which is not PRRS. In addition, the vaccine may comprise certain pharmaceutically or veterinary acceptable adjuvants. One such adjuvant is alpha-tocopherol. Thus, new vaccine compositions, in particular, PRRS virus vaccines comprising PRRSV 94881 may be further improved by addition of adjuvants. Such improvements comprise preparation of the vaccines in combination with adjuvants that enhance the efficacy of the vaccine such that a better clinical response/outcome is seen with the administration of the combination of the adjuvant and the vaccine as compared to administration of the vaccine alone. For example, the vaccine compositions of the invention may comprise a PRRSV 94881 virus vaccine and an adjuvant selected from the group consisting of MCP-1, Haemophilus sonmus fractions, Carbopol® and combinations thereof. In some embodiments, the virus vaccine comprising the PRRSV 94881 virus vaccine, which may be a recombinant subunit vaccine or alternatively may be a live attenuated virus vaccine. An exemplary live vaccine that exists is Ingelvac®PRRS MLV and the PRRSV 94881 may be formulated in a manner similar to Ingelvac®PRRS MLV.

In addition to the above, the vaccine compositions may contain other ingredients so long as the other ingredients do not interfere with the adjuvant properties of the MCP-1, Haemophilus sonmus fractions, Carbapol® or other carbomer or the underlying virus vaccine. Such other ingredients include, for example, binders, colorants, desiccants, antiseptics, wetting agents, stabilizers, excipients, adhesives, plasticizers, tackifiers, thickeners, patch materials, ointment bases, keratin removers, basic substances, absorption promoters, fatty acids, fatty acid ester, higher alcohols, surfactants, water, and buffer agents. Preferred other ingredients include buffer agents, ointment bases, fatty acids, antiseptics, basic substances, or surfactants.

The content or amount of the adjuvants used in the invention may vary and can be determined by taking into consideration, for example, the properties of the PRRS virus vaccine being used, and the dosage form.

The vaccine compositions of the invention may be formulated by any method known in the art of formulation, for example, into liquid preparations, suspensions, ointments, powders, lotions, W/O emulsions, O/W emulsions, emulsions, creams, cataplasms, patches, and gels and is preferably used as medicaments. Thus, according to another aspect of the present invention, there is provided a pharmaceutical composition comprising the above vaccine composition. The vaccine composition according to the present invention, when dermally administered, can significantly induce antibody production. Accordingly, in another embodiment, the vaccine composition of the present invention can be provided as a transdermal preparation.

When the adjuvant and the PRRS virus vaccine are administered to an organism, the clinical outcome of the animal is enhanced. The effective amount of the adjuvant and the immunologically effective amount of the PRRS virus vaccine may be properly determined by a person having ordinary skill in the art by taking into consideration, for example, the type and properties of the antigenic substance, the species of organisms, age, body weight, severity of diseases, the type of diseases, the time of administration, and administration method and further using the amount of an antibody produced against the antigenic substance in the organism as an index.

The PRRS virus vaccine, the adjuvant, or combinations thereof can be administered to organisms by any suitable method selected depending, for example, upon the condition of patients and properties of diseases. Examples of such methods include intraperitoneal administration, dermal administration (for example, subcutaneous injection, intramuscular injection, intradermal injection, and patching), nasal administration, oral administration, mucosa administration (for example, rectal administration, vaginal administration, and corneal administration). Among them, intramuscular administration is preferred.

An exemplary therapeutic dose of PRRSV MLV is about two milliliters (2 mLs). Skilled artisans will recognize that the dosage amount may be varied based on the breed, size, and other physical factors of the individual subject, as well as, the specific formulation of PRRSV MLV and the route of administration. Preferably, the PRRSV MLV is administered in a single dose; however, additional doses may be useful. Again, the skilled artisan will recognize through the present invention that the dosage and number of doses is influenced by the age and physical condition of the subject pig, as well as, other considerations common to the industry and the specific conditions under which the PRRSV MLV is administered.

In certain other embodiments, the vaccine may be a multivalent vaccine that comprises two or more PRRS viruses where at least one of the PRRS viruses is the attenuated 94881 virus deposited under ECACC Accession No. 11012502. The other PRRS viruses may be one or more selected from the group consisting of PRRSV strain deposited under the Accession Numbers Lelystad virus strain (Lelystad Agent (CDI-NL-2.91), or other strains such as those deposited under the Accession Numbers ECACC 04102703, ECACC 04102702, ECACC 04102704, CNCM Accession No. I-1140, CNCM Accession No 1-1387, CNCM Accession No 1-1388, ATCC VR 2332, VR 2385, VR 2386, VR 2429, VR 2474, and VR 2402; CNCM 1-1102, CNCM 1-1140, CNCM 1-1387, CNCM I-1388, or ECACC V93070108 or indeed may be a U.S. strain such as North American PRRS virus, pT7P129A; ATCC deposit VR-2332, ATCC deposit VR-2368; ATCC VR-2495; ATCC VR 2385, ATCC VR 2386, ATCC VR 2429, ATCC VR 2474, and ATCC VR 2402.

The vaccines based on PRRS viruses may be used to vaccinate both piglets and sows. In one aspect of the invention, a particular dose regimen is selected based on the age of the pig and antigen selected for administration. This will permit pigs of any age to receive the most efficacious dose based on the present invention’s discovery that PRRSV infection (from both wild type exposure and vaccination) is cleared much more quickly in older animals. Thus, in some respects, vaccination of older animals is preferred but that vaccination of younger pigs, including those three weeks of age and younger helps to induce active immunity and is still very beneficial. Animal age may be an important factor in PRRS control and may be a factor that impacts vaccination and development of an effective immune response. Thus, age, disease management, animal husbandry, innate, and active immunity are important and need to be considered in control strategies.

The PRRSV vaccine can be administered in any conventional fashion and in some preferred methods the administration is nasally. It is preferred that the administered PRRSV vaccine provide its benefits of treating or reducing the severity of or incidence of PRRSV infection after a single dose, as with Ingelvac PRRS ®, however, if other antigens or combination or multivalent vaccines are selected, it should be understood that they can be administered in their conventional fashion, which may include one or more booster doses after the initial administration. Those of skill in the art will be able to determine appropriate dosing levels based on the PRRSV vaccine selected and the age range of the animal to which the antigen will be administered.

In an advantageous embodiment, the PRRSV vaccine is Ingelvac PRRS® MLV. Accordingly, in an advantageous embodiment, the antigen of PRRSV is the antigen of PRRSV included in Ingelvac PRRS® MLV. A preferred method of immunization or of vaccination consists in the administration of the vaccine according to the invention by systemic administration such as the intramuscular route.

In a particularly advantageous embodiment, the antigen of PCV, the antigen of M. hyo. and the antigen of PRRSV are the antigen of PCV, the antigen of M. hyo. and the antigen of PRRSV included in 3FLEX®.

In a particularly advantageous embodiment, the antigen of Lawsonia intracellularis is lyophilized and dissolved in the 3FLEX® vaccine. In another particularly advantageous embodiment, the antigen of Lawsonia intracellularis included in Enterisol® Ileitis is dissolved in the 3FLEX® vaccine. In an even more particularly advantageous embodiment, the antigen of Lawsonia intracellularis included in Enterisol® Ileitis is dissolved in Ingelvac CircoFLEX®. The volume of the vaccine may be 2 ml.

The invention further contemplates a vaccine that may further comprise one or more antimicrobials. The antimicrobials include, but are not limited to, tiamulin and/or chlortetracycline. In this instance, the dosage of tiamulin may be about 35 g/ton or about 35 ppm and dosage of chlortetracycline may be about 400 g/ton or about 400 ppm.

The combined vaccine of the present invention is advantageously administered intramuscularly, although oral administration is also contemplated.

The present invention also encompasses combinations with antigens from another disease-causing organism in swine. Preferably the other disease-causing organism in swine is selected from the group consisting of: Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani; Coronavirus, preferably Porcine Respiratory Corona virus; Eperythrozoonosis suis; Erysipelothrix rhusiopathiae; Escherichia coli; Haemophilus parasuis, preferably subtypes 1, 7 and 14; Hemagglutinating encephalomyelitis virus; Japanese Encephalitis Virus; Leptospira spp., preferably Leptospira australis, Leptospira canicola, Leptospira grippotyphosa, Leptospira icterohaemorrhagicae, and Leptospira interrogans, Leptospira pomona, Leptospira tarassovi; Mycobacterium spp., preferably M. avium, M. intracellulare and M. bovis; Pasteurella multocida; Porcine cytomegalovirus; Porcine Parvovirus; Pseudorabies virus; Rotavirus; Salmonella spp., preferably S. thyphimurium and S. choleraesuis; Staph. hyicus; Staphylococcus spp, preferably Streptococcus spp., preferably Strep. suis; Swine herpes virus; Swine Influenza Virus; Swine pox virus Vesicular stomatitis virus; Virus of vesicular exanthema of swine; Leptospira Hardjo and/or Mycoplasma hyosynoviae.

The immunogenic preparations of the invention may also be combined with at least one conventional vaccine (attenuated live, inactivated or subunit) or recombinant vaccine (viral vector) directed against at least one porcine pathogen which is different or identical. The invention provides in particular for the combination with adjuvant-containing conventional vaccines (attenuated live, inactivated or subunit). For the inactivated or subunit vaccines, there may be mentioned those containing in particular alumina gel alone or mixed with saponin as adjuvant, or those formulated in the form of an oil-in-water emulsion.

Additionally, the composition may include one or more veterinary-acceptable carriers. As used herein, “a veterinary-acceptable carrier” includes any and all solvents, dispersion media, coatings, adjuvants, stabilizing agents, diluents, preservatives, antibacterial and antifungal agents, isotonic agents, adsorption delaying agents, and the like. In a preferred embodiment, the immunogenic composition comprises PCV3 ORF2 protein or PCV2 ORF2 as provided herewith, preferably in concentrations described above, which is mixed with an adjuvant, preferably CARBOPOL®, and physiological saline.

Those of skill in the art will understand that the composition used herein may incorporate known injectable, physiologically acceptable sterile solutions. For preparing a ready-to-use solution for parenteral injection or infusion, aqueous isotonic solutions, such as e.g. saline or corresponding plasma protein solutions, are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid, which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name CARBOPOL®; (BF Goodrich, Ohio, USA) are particularly appropriate. They are cross-linked with an allyl sucrose or with allyl pentaerythritol. Among them, there may be mentioned CARBOPOL™ 974P, 941P, 934P and 971P. Most preferred is the use of CARBOPOL®, in particular the use of CARBOPOL® 971P, preferably in amounts of about 500 µg to about 5 mg per dose, even more preferred in an amount of about 750 µg to about 2.5 mg per dose and most preferred in an amount of about 1 mg per dose. In particular, a dose of the final composition may include CARBOPOL®or CARBOPOL® 971 in a range from about 750 µg to about 2.5 mg CARBOPOL®. For example, in some embodiments a dose of the final composition may include about 1 mg of CARBOPOL® 971.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314, or muramyl dipeptide among many others.

In other words, the vaccine of the present invention may comprise one or more adjuvant(s). Non-limiting examples for adjuvants are provided throughout the specification.

Furthermore, the vaccine of the present invention may comprise as adjuvant one or more of a polymer of acrylic or methacrylic acid; a copolymer of maleic anhydride and an alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol®; Carbopol® 974P; Carbopol® 934P; Carbopol® 971P; Carbopol® 980; Carbopol® 941P; ImpranFLEX®; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

In a preferred and advantageous embodiment, the vaccines of the present invention comprise one or more carbomer(s).

In a preferred and advantageous embodiment, the vaccines of the present invention comprise Carbopol® and/or ImpranFLEX®. Specific examples of Carbopol® are provided herein.

In a further embodiment, the vaccines of the present invention may comprise a pharmaceutically or veterinarily acceptable carrier.

Preferably, the adjuvant is added in an amount of about 100 µg to about 10 mg per dose. Even more preferably, the adjuvant is added in an amount of about 100 µg to about 10 mg per dose. Even more preferably, the adjuvant is added in an amount of about 500 µg to about 5 mg per dose. Even more preferably, the adjuvant is added in an amount of about 750 µg to about 2.5 mg per dose. Most preferably, the adjuvant is added in an amount of about 1 mg per dose.

Additionally, the composition can include one or more pharmaceutical-acceptable carriers. As used herein, “a pharmaceutical-acceptable carrier” includes any and all solvents, dispersion media, coatings, stabilizing agents, diluents, preservatives, antibacterial and antifungal agents, isotonic agents, adsorption delaying agents, and the like.

According to a further aspect, this immunogenic composition further comprises a pharmaceutical acceptable salt, preferably a phosphate salt in physiologically acceptable concentrations. Preferably, the pH of said immunogenic composition is adjusted to a physiological pH, meaning between about 6.5 and 7.5.

Dosing regimens may be used to improve the economics of swine husbandry. For example, immunogenic compositions, such as vaccines may be administered to sows and/or piglets in an effort to protect sows, piglets, or both.

It is further claimed that the vaccine of the invention is able to protect bred gilts and sows when challenged with Lawsonia intracellularis, PCV, M. hyo. or PRRSV in all or two or at least one trimester during the 114 days of gestation.

It is also claimed that the vaccine is able to significantly reduce the incidence of mummies, stillborns and fetus in vaccinated gilts and sows vaccinated when challenged with Lawsonia intracellularis, PCV, M. hyo. or PRRSV in all or two or at least one trimester during the 114 days of gestation.

A dosing regimen may include vaccinating young sows (i.e., less than or equal to 5 months of age) with at least one dose of an immunogenic composition as described herein prior to breeding. The dose of the immunogenic composition as described herein may be administered intramuscularly as a one (1) mL dose prior to breeding. In some embodiments, one or more doses of vaccine may be given to sows. For example, a first vaccine may be given and followed by a booster vaccine 21 days later and prior to breeding. In some embodiments, sows may be bred in a range from 14 days to 21 days after the booster vaccination. This time frame may allow sows to mount an immune response. Utilizing such a dosing regimen may reduce and/or inhibit the number of mummies at farrowing.

Further, use of a dosing regimen that includes administering an immunogenic composition than includes Lawsonia intracellularis, PCV, M. hyo. or PRRSV may reduce, lessen and /or inhibit lymphadenopathy, lymphoid depletion and/or multinucleated/giant histiocytes in pigs infected with Lawsonia intracellularis, PCV, M. hyo. or PRRSV. Advantageously, the dose is about 2 ml. Described is also a method of immunization which makes it possible to induce an immune response in pigs towards the circoviruses. In particular a method of vaccination which is effective in pigs is described. These methods of immunization and vaccination comprise the administration of one of the preparations or of one of the monovalent or multivalent vaccines as described above. These methods of immunization and vaccination comprise the administration of one or more successive doses of these preparations or vaccines. The preparations and vaccines may be administered, in the context of this method of immunization or of vaccination, by various routes of administration-proposed in the prior art for polynucleotide vaccination, in particular the intramuscular and intradermal routes, and by means of known administration techniques, in particular injections with a syringe having a needle, by liquid jet (Furth et al. Analytical Bioch., 1992, 205: 365-368) or by projection of gold particles coated with DNA (Tang et al. Nature, 1992, 356: 152-154).

This method not only allows for administration to adult pigs, but also to the young and to gestating females; in the latter case, this makes it possible, in particular, to confer passive immunity onto the newborns (maternal antibodies). Preferably, female pigs are inoculated prior to breeding; and/or prior to serving, and/or during gestation. Advantageously, at least one inoculation is done before serving and it is preferably followed by an inoculation to be performed during gestation, e.g., at about mid-gestation (at about 6-8 weeks of gestation) and/or at the end of gestation (at about 11-13 weeks of gestation). Thus, an advantageous regimen is an inoculation before serving and a booster inoculation during gestation. Thereafter, there can be reinoculation before each serving and/or during gestation at about mid-gestation and/or at the end of gestation. Preferably, reinoculations are during gestation.

In a further aspect, the present invention relates to the use of the vaccine of the invention described herein. Furthermore, the present invention relates to methods, wherein the methods comprise the use of the vaccine of the invention described herein.

Accordingly, in one embodiment, the vaccine of the present invention is for use in a method for eliciting a protective immune response in an animal comprising administering said vaccine to the animal.

Accordingly, in one embodiment, the vaccine of the present invention is for use in a method for eliciting a protective immune response in a pig comprising administering said vaccine to the pig. In an advantageous embodiment, the vaccine of the present invention is for use in a method for eliciting a protective immune response in an animal, wherein the vaccine is administered systemically, preferably intramuscularly or intradermally.

In an advantageous embodiment, the vaccine of the present invention is for use in a method for eliciting a protective immune response in an animal, wherein the vaccine is administered as one dose or at least one dose.

The present invention also encompasses that the vaccine of the present invention is for use in a method for eliciting a protective immune response against Lawsonia intracellularis and/or PCV and/or M. hyo. and/or PRRSV in an animal.

In an advantageous embodiment, the vaccine of the present invention is for use in a method for eliciting a protective immune response against Lawsonia intracellularis and M. hyo..

In an advantageous embodiment, the vaccine of the present invention is for use in a method for eliciting a protective immune response against Lawsonia intracellularis and PRRS.

In an advantageous embodiment, the vaccine of the present invention is for use in a method for eliciting a protective immune response against Lawsonia intracellularis and PCV and M. hyo..

In an advantageous embodiment, the vaccine of the present invention is for use in a method for eliciting a protective immune response against Lawsonia intracellularis and PCV and PRRS.

In an advantageous embodiment, the vaccine of the present invention is for use in a method for eliciting a protective immune response against Lawsonia intracellularis and PRRS and M. hyo..

In an advantageous embodiment, the vaccine of the present invention is for use in a method for eliciting a protective immune response against Lawsonia intracellularis and PCV and M. hyo. and PRRSV.

In an advantageous embodiment, the vaccine of the present invention is for use in a method for eliciting a protective immune response against Lawsonia intracellularis and PCV.

In an advantageous embodiment, the vaccine of the present invention is for use in a method for eliciting a protective immune response against Lawsonia intracellularis and PCV, wherein said vaccine comprises live Lawsonia intracellularis, preferably attenuated Lawsonia intracellularis or modified-live Lawsonia intracellularis, and an antigen of PCV.

In an advantageous embodiment, the vaccine of the present invention is for use in a method for eliciting a protective immune response against Lawsonia intracellularis and PCV, wherein said vaccine comprises live Lawsonia intracellularis, preferably attenuated Lawsonia intracellularis or modified-live Lawsonia intracellularis, and a recombinant polypeptide of PCV.

In an advantageous embodiment, the vaccine of the present invention is for use in a method for eliciting a protective immune response against Lawsonia intracellularis and PCV, wherein said vaccine comprises live Lawsonia intracellularis, preferably attenuated Lawsonia intracellularis or modified-live Lawsonia intracellularis, and a recombinant polypeptide of PCV expressed by a PCV ORF2 gene.

In an advantageous embodiment, the vaccine of the present invention is for use in a method for eliciting a protective immune response against Lawsonia intracellularis and PCV, wherein said vaccine comprises the antigen of Lawsonia intracellularis included in Enterisol® Ileitis and an antigen of PCV.

In an advantageous embodiment, the vaccine of the present invention is for use in a method for eliciting a protective immune response against Lawsonia intracellularis and PCV, wherein said vaccine comprises the antigen of Lawsonia intracellularis included in Enterisol® Ileitis and the antigen of PCV included in Ingelvac CircoFLEX® or 3FLEX®.

In an advantageous embodiment, the vaccine of the present invention is for use in a method for eliciting a protective immune response against Lawsonia intracellularis and PCV, wherein said vaccine comprises an antigen of Lawsonia intracellularis and an antigen of PCV, and wherein said vaccine is administered systemically, preferably intramuscularly or intradermally.

In an advantageous embodiment, the vaccine of the present invention is for use in a method for eliciting a protective immune response against Lawsonia intracellularis and PCV, wherein said vaccine comprises the antigen of Lawsonia intracellularis included in Enterisol® Ileitis and the antigen of PCV included in Ingelvac CircoFLEX® or 3FLEX®, and wherein said vaccine is administered systemically, preferably intramuscularly or intradermally.

In one embodiment, the vaccine of the present invention is for use in a method for immunizing an animal against a clinical disease caused by at least one pathogen in said animal, wherein said vaccine fails to cause clinical signs of infection but is capable of inducing an immune response that immunizes the animal against pathogenic forms of said at least one pathogen.

In an advantageous embodiment, the vaccine of the present invention is for use in a method for immunizing an animal against a clinical disease caused by Lawsonia intracellularis and PCV in said animal, wherein said vaccine fails to cause clinical signs of infection but is capable of inducing an immune response that immunizes the animal against pathogenic forms of said pathogens.

In one embodiment, the vaccine of the present invention is for use in a method for eliciting a protective immune response, wherein the protective immune response against Lawsonia intracellularis is for reducing intestinal lesions in an animal, in comparison to an animal of a non-immunized control group of the same species.

Accordingly, in an advantageous embodiment, the vaccine of the present invention is for use in a method for eliciting a protective immune response, wherein the protective immune response against Lawsonia intracellularis is for reducing intestinal lesions in an animal, in comparison to an animal of a non-immunized control group of the same species, wherein said vaccine comprises the antigen of Lawsonia intracellularis included in Enterisol® Ileitis and the antigen of PCV included in Ingelvac CircoFLEX® or 3FLEX®.

The intestinal lesions may be ileum lesions. The intestinal lesions and/or ileum lesions may be macroscopic lesions and/or microscopic lesions.

In one embodiment, the vaccine of the present invention is for use in a method for eliciting a protective immune response, wherein the protective immune response against Lawsonia intracellularis is for reducing fecal shedding of an animal, in comparison to an animal of a non-immunized control group of the same species.

In an advantageous embodiment, the vaccine of the present invention is for use in a method for eliciting a protective immune response, wherein the protective immune response against Lawsonia intracellularis is for reducing fecal shedding of an animal, in comparison to an animal of a non-immunized control group of the same species, wherein said vaccine comprises the antigen of Lawsonia intracellularis included in Enterisol® Ileitis and the antigen of PCV included in Ingelvac CircoFLEX® or 3FLEX®.

In one embodiment, the vaccine of the present invention is for use in a method for eliciting a protective immune response, wherein the protective immune response against Lawsonia intracellularis is for increasing the average daily weight gain of an animal, in comparison to an animal of a non-immunized control group of the same species.

In an advantageous embodiment, the vaccine of the present invention is for use in a method for eliciting a protective immune response, wherein the protective immune response against Lawsonia intracellularis is for increasing the average daily weight gain of an animal, in comparison to an animal of a non-immunized control group of the same species, wherein said vaccine comprises the antigen of Lawsonia intracellularis included in Enterisol® Ileitis and the antigen of PCV included in Ingelvac CircoFLEX® or 3FLEX®.

The present invention further encompasses a method for eliciting a protective immune response against Lawsonia intracellularis and/or PCV and/or M. hyo. and/or PRRSV in an animal comprising administering to the animal the vaccine of the present invention.

The present invention also encompasses a method for eliciting a protective immune response against Lawsonia intracellularis and PCV in an animal comprising administering to the animal the vaccine of the present invention.

The present invention also encompasses a method for eliciting a protective immune response against Lawsonia intracellularis and PCV in a pig comprising administering to the pig the vaccine of the present invention.

The present invention also encompasses a method of immunizing an animal against a clinical disease caused by at least one pathogen in said animal, said method comprising the step of administering to the animal the vaccine of the present invention, wherein said vaccine fails to cause clinical signs of infection but is capable of inducing an immune response that immunizes the animal against pathogenic forms of said at least one pathogen.

The present invention also encompasses a method of immunizing an animal against a clinical disease caused by Lawsonia intracellularis and PCV in said animal, said method comprising the step of administering to the animal the vaccine of the present invention, wherein said vaccine fails to cause clinical signs of infection but is capable of inducing an immune response that immunizes the animal against pathogenic forms of said pathogens.

The present invention also encompasses a method of immunizing a pig against a clinical disease caused by Lawsonia intracellularis and PCV in said pig, said method comprising the step of administering to the animal the vaccine of the present invention, wherein said vaccine fails to cause clinical signs of infection but is capable of inducing an immune response that immunizes the pig against pathogenic forms of said pathogens.

The present invention further encompasses the use of the vaccine of the present invention in the preparation of a composition for inducing a protective immune response against Lawsonia intracellularis and/or PCV and/or M. hyo. and/or PRRSV.

In an advantageous embodiment, the use of the vaccine of the present invention in the preparation of a composition for inducing a protective immune response against Lawsonia intracellularis and PCV.

In an advantageous embodiment, the use of the vaccine of the present invention in the preparation of a composition for inducing a protective immune response against Lawsonia intracellularis and PCV and M hyo. and PRRS.

The present invention further encompasses the use of the vaccine of the present invention for a method for inducing a protective immune response against Lawsonia intracellularis and/or PCV and/or M. hyo. and/or PRRSV.

In an advantageous embodiment, the use of the vaccine of the present is for a method for inducing a protective immune response against Lawsonia intracellularis and PCV.

In an advantageous embodiment, the use of the vaccine of the present is for a method for inducing a protective immune response against Lawsonia intracellularis and PCV and M hyo. and PRRS.

The vaccine of the present invention may preferably be administered as a single dose, i.e. one-shot administration.

Accordingly, in one embodiment, the vaccine of the present invention is formulated and/or packaged for a single dose or one-shot administration.

In one embodiment, the vaccine of the present invention is formulated and/or packaged for a multi-dose regimen, preferably a two-dose regimen.

In one embodiment, the vaccine of the present invention is in a dosage form, wherein said dosage form is delivered from a container containing a larger amount of said vaccine and wherein a dosage form of said vaccine is capable of being delivered from said container. Said container may contain at least 10, at least 50, at least 100, at least 150, at least 200 or at least 250 doses of said vaccine.

Usually, the disease caused by Lawsonia intracellularis porcine proliferative enteropathy (PPE) can be controlled using a commercial live vaccine (Enterisol® Ileitis) which is given orally by drench or in drinking water. It is required that three days before, on the day of vaccination and three days after vaccination animals vaccinated with Enterisol® Ileitis do not receive any antibiotic treatment effective against Lawsonia intracellularis.

The inventors surprisingly and unexpectedly found that a live Lawsonia intracellularis vaccine is effective despite simultaneous/concomitant antibiotic treatment of the animal when administered intramuscularly.

Accordingly, it is envisaged herein that the vaccine of the present invention can be administered to the animal despite a simultaneous/concomitant antibiotic treatment of the animal. When the vaccine of the present invention is administered despite simultaneous/concomitant antibiotic treatment of the animal, the administration route is preferably systemic administration.

Accordingly, the present invention encompasses the vaccine of the present invention for use in a method for eliciting a protective immune response in an animal comprising administering said vaccine to the animal, wherein the animal is simultaneously/concomitantly treated with one or more antibiotic(s).

Furthermore, the present invention encompasses the vaccine of the present invention for use in a method for eliciting a protective immune response in a pig comprising administering said vaccine to the pig, wherein the pig is simultaneously/concomitantly treated with one or more antibiotic(s). The phrase “simultaneously/concomitantly treated with one or more antibiotic(s)” as used herein means that the animal/pig received antibiotic treatment (i.e. one or more antibiotics were administered to the animal/pig) three days before vaccination, two days before vaccination and/or one day before vaccination. Said phrase can also mean that the animal/pig received an antibiotic treatment and vaccination on the same day. Said phrase can also mean that the animal/pig will receive an antibiotic treatment one day, two days and/or three days after vaccination.

The term “antibiotic” is well known in the art and is used herein in the broadest sense. The term “antibiotic” as used herein may refer to compounds that have an adverse effect on bacteria. Nonlimiting examples of antibiotics include beta-lactams (e.g., Penicillin VK, Penicillin G, Amoxicillin trihydrate), nitroimidazoles, macrolides (e.g., Tylosin tartrate, Erythromycin, Azithromycin, and Clarithromycin), tetracyclines, glycopeptides (e.g., Vancomycin), pleuromutilins and fluoroquinolones.

Preferably, the antibiotic Denagard® (tiamulin) or CTC (chlortetracycline) or a combination thereof is used for the antibiotic treatment.

Preferably, the antibiotics are administered at a dosage of 35 g/ton of Denagard® (tiamulin) and 400 g/ton of CTC (chlortetracycline) for a total period of two weeks.

Accordingly, the present invention encompasses the vaccine of the present invention for use in a method for eliciting a protective immune response in an animal comprising administering said vaccine to the animal, wherein the animal is simultaneously/concomitantly treated with one or more antibiotic(s), and wherein said vaccine comprises an antigen of Lawsonia intracellularis and an antigen of PCV.

In an advantageous embodiment, the vaccine of the present invention is for use in a method for eliciting a protective immune response in an animal comprising administering said vaccine to the animal, wherein the animal is simultaneously/concomitantly treated with one or more antibiotic(s), wherein said vaccine comprises live Lawsonia intracellularis and an antigen of PCV, and wherein the vaccine is administered systemically, preferably intramuscularly.

Furthermore, the present invention encompasses the vaccine of the present invention for use in a method for eliciting a protective immune response in an animal comprising administering said vaccine to the animal, wherein the animal is simultaneously/concomitantly treated with one or more antibiotic(s), and wherein said vaccine comprises the antigen of Lawsonia intracellularis included in Enterisol® Ileitis and the antigen of PCV included Ingelvac CircoFLEX® or 3FLEX®. Furthermore, the present invention encompasses the vaccine of the present invention for use in a method for eliciting a protective immune response in an animal comprising administering said vaccine to the animal, wherein the animal is simultaneously/concomitantly treated with Denagard® (tiamulin) and/or CTC (chlortetracycline), and wherein said vaccine comprises the antigen of Lawsonia intracellularis included in Enterisol® Ileitis and the antigen of PCV included Ingelvac CircoFLEX® or 3FLEX®.

In an advantageous embodiment, the vaccine of the present invention is for use in a method for eliciting a protective immune response in an animal comprising administering said vaccine to the animal, wherein the animal is simultaneously/concomitantly treated with Denagard® (tiamulin) and/or CTC (chlortetracycline), wherein said vaccine comprises the antigen of Lawsonia intracellularis included in Enterisol® Ileitis and the antigen of PCV included Ingelvac CircoFLEX® or 3FLEX®, and wherein the vaccine is administered systemically, preferably intramuscularly.

Although the present invention and its advantages have been described in detail, it should be understood that various changes, substitutions and alterations can be made herein without departing from the spirit and scope of the invention as defined in the appended claims.

Certain aspects of the present invention will now be described by way of numbered paragraphs.
1. A vaccine suitable for use as a porcine vaccine comprising an immunogenic Lawsonia intracellularis component, an immunogenic porcine circovirus (PCV) component, an immunogenic Mycoplasma hyopneumoniae (M. hyo.) component, and an immunogenic porcine respiratory and reproductive syndrome virus (PRRSV) component.
2. The vaccine of paragraph 1 wherein the vaccine comprises one or more adjuvants.
3. The vaccine of paragraph 2 wherein the one or more adjuvants comprises one or more of a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol; Carbopol 974P; Carbopol 934P; Carbopol 971P; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide
4. A vaccine according to any one of the preceding paragraphs wherein the immunogenic Lawsonia intracellularis component is a Lawsonia intracellularis vaccine.
5. A vaccine according to any one of the preceding paragraphs wherein the immunogenic Lawsonia intracellularis component is a live vaccine.
6. A vaccine according to any one of the preceding paragraphs wherein the Lawsonia intracellularis component is an attenuated vaccine.
7. The vaccine of any one of the preceding paragraphs, wherein the Lawsonia intracellularis component has a dosage of 10³ to 10⁹ bacteria/Kg of body weight or about 10⁵ to 10⁷ bacteria/Kg of body weight.
8. The vaccine of any one of the preceding paragraphs, wherein the Lawsonia intracellularis component has a dosage of 1 × 10⁵ to 1 × 10⁷ of Lawsonia intracellularis bacteria.
9. The vaccine of any one of the preceding paragraphs, wherein the Lawsonia intracellularis component is lyophilized.
10. The vaccine of any one of the preceding paragraphs, wherein the Lawsonia intracellularis vaccine further comprises an adjuvant.
11. The vaccine of the preceding paragraph, wherein the adjuvant is ImpranFLEX®.
12. The vaccine of any one of the preceding paragraphs, wherein the Lawsonia intracellularis component is an Enterisol® Ileitis vaccine.
13. A vaccine according to any one of the preceding paragraphs wherein the immunogenic porcine circovirus (PCV) component is a porcine circovirus (PCV) vaccine.
14. The vaccine of any one of the preceding paragraphs, wherein the PCV is PCV1.
15. The vaccine of any one of the preceding paragraphs, wherein the PCV is PCV2.
16. The vaccine of any one of the preceding paragraphs, wherein the PCV is PCV3.
17. The vaccine of any one of the preceding paragraphs, wherein the PCV is PCV2 and PCV3.
18. The vaccine of any one of the preceding paragraphs, wherein the PCV component is a recombinant PCV vaccine.
19. The vaccine of any one of the preceding paragraphs, wherein the recombinant PCV component is or comprises or is expressed by a PCV ORF gene, such as a protein expressed by the PCV ORF gene.
20. The vaccine of any one of the preceding paragraphs, wherein the recombinant PCV component is or comprises or is expressed by a PCV ORF gene, such as a protein expressed by the PCV ORF gene, and wherein the PCV ORF gene encodes a PCV ORF2 gene.
21. The vaccine of any one of the preceding paragraphs, wherein the recombinant PCV component is or comprises or is expressed by a PCV ORF gene, such as a protein expressed by the PCV ORF gene, wherein the PCV ORF gene encodes a PCV ORF2 gene, and wherein the PCV ORF2 gene is a PCV2 ORF2 gene.
22. The vaccine of any one of the preceding paragraphs, wherein the PCV component is or comprises a recombinant PCV ORF2 protein.
23. The vaccine of any one of the preceding paragraphs, wherein the PCV component is or comprises a recombinant PCV ORF2 protein, and wherein the vaccine has a dosage of about 2 µg to about 400 µg of recombinant PCV ORF2 protein.
24. The vaccine of any one of the preceding paragraphs, wherein the PCV component is or comprises DNA.
25. The vaccine of any one of the preceding paragraphs, wherein the PCV component is or comprises DNA, and wherein the DNA is in an amount of between about 10 µg and about 2000 µg, and preferably between about 50 µg and about 1000 µg.
26. The vaccine of any one of the preceding paragraphs, wherein the PCV component is or has been expressed in a baculovirus cell.
27. The vaccine of any one of the preceding paragraphs, wherein the PCV component further comprises an adjuvant.
28. The vaccine of any one of the preceding paragraphs, wherein vaccine or one of the vaccine components comprises an adjuvant, and wherein the adjuvant is CARBOPOL™.
29. The vaccine of any one of the preceding paragraphs, wherein the PCV component is Ingelvac CircoFLEX®.
30. A vaccine according to any one of the preceding paragraphs wherein the immunogenic Mycoplasma hyopneumoniae (M. hyo) component is a Mycoplasma hyopneumoniae (M. hyo) vaccine.
31. The vaccine of any one of the preceding paragraphs, wherein the M. hyo. component is a supernatant and/or a bacterin.
32. The vaccine of the preceding paragraph, wherein the M. hyo. component is a bacterin.
33. The vaccine of any one of the preceding paragraphs, wherein the dosage of the M. hyo. component is about 2 ml of supernatant and/or bacterin.
34. The vaccine of any one of the preceding paragraphs, wherein the M. hyo. component is Ingelvac MycoFlex®.
35. A vaccine according to any one of the preceding paragraphs wherein the immunogenic porcine respiratory and reproductive syndrome virus (PRRSV) component is a porcine respiratory and reproductive syndrome virus (PRRSV) vaccine.
36. The vaccine of any one of the preceding paragraphs, wherein the PRRSV component is a live vaccine.
37. The vaccine of the preceding paragraph, wherein PRRSV component is an attenuated vaccine.
38. The vaccine of the two preceding paragraphs, wherein PRRSV component is a modified live vaccine.
39. The vaccine of any one of the preceding paragraphs, wherein the PRRSV component has a dosage of about 10¹ to about 10⁷ viral particles per dose, preferably about 10³ to about 10⁵ particles per dose, more preferably about 10⁴ to about 10⁵ particles per dose.
40. The vaccine of any one of the preceding paragraphs, wherein the PRRSV component is lyophilized.
41. The vaccine of the preceding paragraph, wherein the lyophilized PRRSV component is or has been reconstituted in 2 ml of solvent for administration.
42. The vaccine of any one of the preceding paragraphs, wherein the PRRSV component is Ingelvac® PRRS MLV.
43. The vaccine of any one of the preceding paragraphs, wherein the PCV component, the M. hyo. component and the PRRSV component is or is derived from a 3FLEX® vaccine.
44. The vaccine of any one of the preceding paragraphs, wherein the PCV component, the M. hyo. component and the PRRSV component is or is derived from a 3FLEX® vaccine, and wherein the Lawsonia intracellularis component is lyophilized and dissolved in the 3FLEX® vaccine.
45. The vaccine of the two preceding paragraphs, wherein the Lawsonia intracellularis component is an Enterisol® Ileitis.
46. The vaccine of any one of the preceding paragraphs, wherein volume of the vaccine is from about 0.5 ml to about 4 ml.
47. The vaccine of any one of the preceding paragraphs, wherein volume of the vaccine is about 2 ml.
48. The vaccine of any one of the preceding paragraphs further comprising a pharmaceutically or veterinarily acceptable carrier.
49. The vaccine of any one of the preceding paragraphs further comprising an adjuvant.
50. The vaccine of the preceding paragraph, wherein the adjuvant is ImpranFLEX®.
51. The vaccine of any one of the preceding paragraphs, wherein the vaccine is in a form for oral administration.
52. The vaccine of any one of the preceding paragraphs, wherein the vaccine is in a form for oral administration via either drinking water or an oral drench.
53. The vaccine of any one of the preceding paragraphs, wherein the vaccine is in a form for intramuscular administration.
54. A vaccine according to any one of the preceding paragraphs wherein the vaccine is formulated and/or packaged for a single dose or one shot administration.
55. A vaccine according to any one of the preceding paragraphs wherein the vaccine is formulated and/or packaged for a multi-dose regimen.
56. A vaccine according to any one of the preceding paragraphs wherein the vaccine is formulated and/or packaged for a two-dose regimen.
57. The vaccine of any one of the preceding paragraphs, wherein the vaccine is in a dosage form; and wherein said dosage form is delivered from a container containing a larger amount of said vaccine and wherein a dosage form of said vaccine is capable of being delivered from said container.
58. The vaccine of any one of the preceding paragraphs, wherein the vaccine is in a dosage form; and wherein said dosage form is delivered from a container containing a larger amount of said vaccine and wherein a dosage form of said vaccine is capable of being delivered from said container; and wherein said container contains at least 10 doses of said composition.
59. The vaccine of any one of the preceding paragraphs, wherein the vaccine is in a dosage form; and wherein said dosage form is delivered from a container containing a larger amount of said vaccine and wherein a dosage form of said vaccine is capable of being delivered from said container; and wherein said container contains at least 50 doses of said composition.
60. The vaccine of any one of the preceding paragraphs, wherein the vaccine is in a dosage form; and wherein said dosage form is delivered from a container containing a larger amount of said vaccine and wherein a dosage form of said vaccine is capable of being delivered from said container; and wherein said container contains at least 100 doses of said composition.
61. The vaccine of any one of the preceding paragraphs, wherein the vaccine is in a dosage form; and wherein said dosage form is delivered from a container containing a larger amount of said vaccine and wherein a dosage form of said vaccine is capable of being delivered from said container; and wherein said container contains at least 150 doses of said composition.
62. The vaccine of any one of the preceding paragraphs, wherein the vaccine is in a dosage form; and wherein said dosage form is delivered from a container containing a larger amount of said vaccine and wherein a dosage form of said vaccine is capable of being delivered from said container; and wherein said container contains at least 200 doses of said composition.
63. The vaccine of any one of the preceding paragraphs, wherein the vaccine is in a dosage form; and wherein said dosage form is delivered from a container containing a larger amount of said vaccine and wherein a dosage form of said vaccine is capable of being delivered from said container; and wherein said container contains at least 250 doses of said composition.
64. The vaccine of any one of the preceding paragraphs for use in eliciting an immune response or an immunological response or a protective immune response or a protective immunological response in an animal.
65. The vaccine of any one of the preceding paragraphs for use in eliciting an immune response or an immunological response or a protective immune response or a protective immunological response in an animal, wherein the animal is a porcine animal.
66. The vaccine of any one of the preceding paragraphs for the use according to any one of the previous two paragraphs wherein the use is for eliciting an immune response or an immunological response or a protective immune response or a protective immunological response against Lawsonia intracellularis, PCV, M. hyo. and PRRSV in the animal.
67. The vaccine of any one of the preceding paragraphs for the use according to any one of the previous three paragraphs, wherein the vaccine is administered orally.
68. The vaccine of any one of the preceding paragraphs for the use according to any one of the previous four paragraphs, wherein the vaccine is administered orally via drinking water or an oral drench.
69. The vaccine of any one of the preceding paragraphs for the use according to any one of the previous five paragraphs, wherein the vaccine is administered intramuscularly.
70. The vaccine of any one of the preceding paragraphs for the use according to any one of the previous six paragraphs, wherein the vaccine is administered as one dose.
71. The vaccine of any one of the preceding paragraphs for the use according to any one of the previous seven paragraphs, wherein the vaccine is administered as one dose, and wherein said one dose elicits an immune response or an immunological response or a protective immune response or a protective immunological response against Lawsonia intracellularis, PCV, M. hyo. and PRRSV in the animal.
72. The vaccine of any one of the preceding paragraphs for the use according to any one of the previous eight paragraphs, wherein the vaccine is administered as at least one dose.
73. The vaccine of any one of the preceding paragraphs for the use according to any one of the previous nine paragraphs, wherein the vaccine is administered as at least one dose, and wherein said one dose elicits an immune response or an immunological response or a protective immune response or a protective immunological response against Lawsonia intracellularis, PCV, M. hyo. and PRRSV in the animal.
74. The vaccine of any one of the preceding paragraphs for the use according to any one of the previous ten paragraphs, wherein the vaccine is administered in one dose to a porcine animal.
75. The vaccine of any one of the preceding paragraphs for the use according to any one of the previous eleven paragraphs, wherein the vaccine is administered in only one dose to a porcine animal.
76. The vaccine of any one of the preceding paragraphs for the use according to any one of the previous twelve paragraphs, wherein the vaccine is administered in at least one dose to a porcine animal.
77. A method for eliciting an immune response or an immunological response or a protective immune or immunological response against Lawsonia intracellularis, PCV, M. hyo. and PRRSV in an animal comprising administering to the animal the vaccine of any one of the preceding paragraphs.
78. A method of immunizing an animal against a clinical disease caused by at least one pathogen in said animal, said method comprising the step of administering to the animal the vaccine of any one of the preceding paragraphs, wherein said immunogenic composition fails to cause clinical signs of infection but is capable of inducing an immune response that immunizes the animal against pathogenic forms of said at least one pathogen.
79. Use of a vaccine of any one of the preceding paragraphs for use in the preparation of a composition for inducing an immunological or immune response or a protective immune or immunological response against Lawsonia intracellularis, PCV, M. hyo. and PRRSV or for use in a method for inducing an immunological or immune response or a protective immune or immunological response against Lawsonia intracellularis, PCV, M. hyo. and PRRSV.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

The present invention will be further illustrated in the following Examples which are given for illustration purposes only and are not intended to limit the invention in any way.

### EXAMPLES

### Example 1: Efficacy of Enterisol® Ileitis Given Intramuscularly and With the Presence of Antimicrobials Study Design

### Objective

The primary objective of the Example is to evaluate the efficacy of Enterisol® Ileitis combined with 3FLEX® vaccine when injected intramuscularly into pigs that is challenged with a gut homogenate containing virulent Lawsonia intracellularis, the causative agent of PPE. The secondary objective is to evaluate the efficacy of this vaccine combination when administered to pigs given concomitant antimicrobial treatment.

### Justification

PPE can be controlled using a commercial live vaccine (Enterisol® Ileitis) which is given orally by drench or in drinking water. It is required that three days before, on the day of vaccination and three days after vaccination animals vaccinated with Enterisol® Ileitis do not receive any antibiotic treatment effective against Lawsonia. While there is a trend to reduce use of antibiotics, this is still a significant hurdle to overcome on many farms. Using a different route of application might allow vaccine efficacy in the presence of antibiotic treatment, which would facilitate vaccine use. At the same time, the industry has a desire to reduce the number of injections received by pigs, a better understanding of the efficacy of Enterisol® Ileitis when injected in combination with 3FLEX® vaccine would provide insight into the feasibility of developing a combined vaccine against Lawsonia, PCV2, M. hyo., and PRRSV. In this study, vaccination of pigs by the intramuscular route will be investigated for Enterisol® Ileitis combined with 3FLEX® followed with challenge three weeks later with a gut homogenate containing virulent Lawsonia intracellularis.

### Description of Overall Design

One hundred and twelve weaned 21 day (+/- 2 days) old pigs are obtained from a source known to have no clinical history of ileitis. In addition, pigs used in this trial will be fecal qPCR negative as well as serum antibody negative for Lawsonia intracellularis. Pigs are randomly assigned to four groups, three with 24 animals per group, one with 16; blocking by weight, litter and sex. The treatment groups are: positive challenge control (PC); Enterisol® Ileitis given IM with 3®FLEX vaccine (EIIM) and Enterisol® Ileitis given IM with 3®FLEX vaccine with concomitant antimicrobial administration (EIIMATB). A negative control group of 16 pigs is not challenged but is compared to the PC group to evaluate Lawsonia challenge alone. This negative control group is euthanized at the time of study termination along with the other treatment groups. Treatment group EIIMATB is the only group to receive antimicrobials and is given the label dosage of 35 g/ton of Denagard® (tiamulin) and 400 g/ton of CTC (chlortetracycline) for a total period of two weeks. Antimicrobial treatment begins (D-7) after an adaptation period of five days to allow for pigs to recover from feed intake loss due to weaning. After one week of Denagard® CTC in feed, the EIIMATB group is vaccinated IM with a 2 ml dose of Enterisol® Ileitis combined with 3FLEX vaccine. The lyophilized form of Enterisol® Ileitis vaccine is rehydrated using the 3FLEX vaccine in a manner that results in one 2 ml dose of the Enterisol® Ileitis in each 2 ml dose of 3FLEX. This means that a 2 ml dose of the resulting experimental “4FLEX” vaccine contains the appropriate amounts of all four antigens. All treatment groups are vaccinated at the same time (DO). Retention samples of vaccine left over after vaccination will be stored at -80° C. Following vaccination, animals are observed for injection site lesions and any other adverse reactions. Observations are recorded. The positive and negative challenge control groups are only vaccinated with 3FLEX and not given any Lawsonia antigen. After a period of 28 days (4 weeks) to allow for immunity to develop, all animals are challenged orally with a mucosal homogenate containing Lawsonia intracellularis containing a target of 10⁸⁻⁹ organisms per pig. This mucosal homogenate will be sequenced by next generation sequencing to investigate its complete content and for quantitative PCR to quantify Lawsonia intracellularis. Gut homogenate material should be free of other pathogens including Salmonella, PRRSV and Brachyspira species. At time of challenge, all pigs are weighed to allow for a measure of weight gain pre and post challenge. Following challenge, all animals are evaluated daily for clinical signs of altered feces, altered body condition and behavior until termination of the study.

The study is terminated at 21 days post challenge, when all animals are euthanized and weighed again. At necropsy, macroscopic lesions are measured and evaluated in all portions of the intestinal tract, terminal ileum samples as well as any additional affected tissue will be collected in formalin to measure microscopic lesions.

Microscopic lesions are estimated by immunohistochemistry (IHC) and by hematoxylin and eosin (H&E) staining to measure proliferative lesions. Blood and fecal samples are collected from all animals at time of vaccination, time of challenge and weekly there after until necropsy for Lawsonia intracellularis serology and qPCR, respectively. Fecal samples are collected by digital insertion changing gloves between animals or by fecal loop. If fecal samples are collected by fecal loop, these must not be re-used and a different fecal loop must be used for each animal. All blood and fecal samples should be aliquoted and only one aliquot submitted for serology and fecal PCR for Lawsonia. All tubes with samples are labeled with the date of collection, day of study, and pig ID number.

### Experimental Unit Each Individual Pig

### Justification for Number of Replicates

Power Calculation: Assuming an incidence rate in the challenge control group of at least 75%, 21 animals per treatment group are expected to provide approximately 80% power to detect a difference of 40 percentage points between the treatment and the control for a two-sided test using a=0.05. A total number of 24 animals per treatment group are used to allow for potential fallout as well as accepting a power level slightly higher than 80%.

### Method for Randomization

Animals are blocked per weight, sex and litter; a random number generator is used to assign pigs per treatment.

### Levels and Description of Blinding

Treatments are blinded for evaluation of lesions, fecal shedding and serology. Blinding is also performed for statistical analysis.

### Diagnostic Details and Requirements

Following the time points of vaccination and challenge, all study animals have weekly blood and fecal samples collected. This equals approximately 560 blood and fecal samples (112 animals × 5 samplings). All samples are submitted for diagnosis. At least one aliquot of each sample is stored at -80° C. until termination of the study.

Production Phase: Nursery phase of production.

Animal Gender: Barrows and gilts equally distributed between treatment groups as possible.

### Inclusion/Exclusion and Post-Inclusion Removal Criteria

Inclusion Criteria at Study Initiation: Commercially produced group of pigs of normal health status at D(-7). Exclusion Criteria at Study Initiation: Pigs that are clinically ill or unthrifty at D(-7).

### Exclusion Removal Criteria During Study

If welfare or illness concerns arise, the primary investigator and site veterinarian and/or designee evaluates and determines the best course of action which may include euthanasia.

### Example 2: Efficacy of Enterisol® Ileitis Given Intramuscularly and With the Presence of Antimicrobials Final Study Report

This study evaluated the efficacy of the intramuscular route of administration of Enterisol® Ileitis and when combined with Ingelvac®3FLEX vaccine in protecting against Lawsonia intracellularis, the causative agent of porcine proliferative enteropathy (PPE). The potential interference of antimicrobials to vaccine efficacy was also evaluated.

PPE was successfully reproduced in this study as measured by characteristic macroscopic lesions, microscopic lesions, fecal shedding of L. intracellularis, seroconversion to L. intracellularis, clinical signs and impact on production performance. The intramuscular combination of Enterisol® Ileitis with 3FLEX®, “4FLEX”, led to a meaningful and significant reduction in macroscopic lesion severity, microscopic lesion severity, clinical diarrhea score and fecal shedding of L. intracellularis. An increase in average daily weight gain was also observed in this treatment group compared to non-vaccinated controls. These results indicate that the intramuscular route and mixing of Enterisol® Ileitis with 3FLEX® vaccine are a suitable and efficacious option for the prevention of PPE. It was also observed that this vaccine combination was safe and no adverse events or injection site reactions were observed.

When antimicrobials were administered during vaccination, a change was noted to some parameters evaluated in this study. While a reduction in macroscopic and microscopic lesions was observed in the group that received antimicrobials (EIIMATB) relative to non-vaccinated challenged controls, these levels did not reach statistical significance and were numerically increased as compared to the same treatment without antimicrobials (EIIM). The group that was vaccinated while receiving antimicrobials (EIIMATB) however was the vaccinated group with greatest weight gain following challenge, which was significantly increased compared to non-vaccinated controls. A significant reduction in the incidence of altered clinical diarrhea score was also observed. These results indicate that antimicrobials may possibly interfere with vaccine efficacy, yet a considerable level of protection was still conferred when vaccinating in the presence of the tested antimicrobials.

The primary objective of the study was to evaluate the efficacy of the intramuscular route of administration of Enterisol® Ileitis combined with 3FLEX® vaccine in protecting pigs against Lawsonia intracellularis, the causative agent of porcine proliferative enteropathy (PPE).

The second objective was to evaluate the efficacy of this vaccine combination when administered to pigs receiving concomitant antimicrobial treatment.

Vaccine efficacy was determined by the reduction of gross and microscopic intestinal lesions. Other variables of interest including growth performance, clinical signs and fecal shedding were also evaluated.

PPE can be controlled using a commercial live vaccine (Enterisol® Ileitis) which is given orally by drench or in drinking water. It is required that three days before, on the day of vaccination and three days after vaccination animals vaccinated with Enterisol® Ileitis do not receive any antibiotic treatment effective against Lawsonia. While there is a trend to reduce use of antibiotics, this is still a significant hurdle to overcome on many farms. Using a different route of application might allow for vaccine efficacy in the presence of antibiotic treatment, which would facilitate vaccine use. At the same time, the industry has a desire to reduce the number of injections received by pigs, a better understanding of the efficacy of Enterisol® Ileitis when injected in combination with 3FLEX® vaccine would provide insight into the feasibility of developing a combined vaccine against Lawsonia, PCV2, Mycoplasma hyopneumoniae, and PRRSV. In this study, vaccination of pigs by the intramuscular route was investigated for Enterisol® Ileitis combined with 3FLEX® followed with challenge four weeks later with a gut homogenate containing virulent Lawsonia intracellularis.

### Design Considerations

21 day (+/- 2 days) old pigs were obtained from a source known to have no clinical history of ileitis. Pigs were be randomly assigned to four groups, three with 24 animals per group, one with 16. The treatment groups were: positive challenge control (PC); Enterisol® Ileitis given IM with 3®FLEX vaccine (EIIM) and Enterisol® Ileitis given IM with 3®FLEX vaccine with concomitant antimicrobial administration (EIIMATB). A negative control (NC) group of 16 pigs was not challenged, and served the purpose of evaluating the severity of challenge in the PC group. Treatment group EIIMATB was the only group that received antimicrobials and was given the label dosage of 35 g/ton of Denagard® (tiamulin) and 400 g/ton of CTC (chlortetracycline) for a total period of two weeks. Antimicrobial treatment began (D-7) after an adaptation period of 13 days to allow for pigs to recover from feed intake loss due to weaning. After one week of Denagard® CTC in feed, the EIIMATB group was vaccinated IM with a 2 ml dose of Enterisol® Ileitis combined with 3®FLEX vaccine. All treatment groups were vaccinated at the same time (D0). After a period of 28 days (4 weeks) to allow for immunity to develop, all animals were challenged orally with a mucosal homogenate containing Lawsonia intracellularis. The study was terminated at 21 days post challenge, when all animals were euthanized, lesions were scored and samples were collected. Further design details are in the below tables.

Study design

| Group ID | Group Description | # | Lawsonia Vaccine Dose | Lawsonia Vaccine Route | Challenge |
|---|---|---|---|---|---|
| NC | Un-vaccinated & Un-challenged Controls (Negative Control) | 16 | N/A | N/A | No |
| PC | Un-vaccinated & Challenged | 24 | N/A | N/A | Yes |
| | Controls (Positive Control) | | | | |
| EIIM | Enterisol® Ileitis IM + 3FLEX (“4FLEX”) | 24 | 2ml | IM | Yes |
| EIIMATB | Enterisol® Ileitis IM + 3FLEX + Tiamulin/Chlortetracyline (“4FLEX” with antimicrobial) | 24 | 2ml | IM | Yes |

Schedule of Events

| | | |
|---|---|---|
| Trial Day | Date | Event and Recording Form (code in parenthesis) |
| Day (-20) | | Pigs arrive at GBI; Perform Animal Health Examination (AHE_1.0); Daily General Health Examination until challenge date (GHO_1.0); Weigh All Animals (BWR_1.0); Assign animals to treatments and pens blocking for weight, sex and litter |
| Day (-7) | | Begin antibiotic treatment in feed for EIIMATB group (BPT 1.0) |
| Day 0 | | Vaccinate all animals (PDR_1.1), follow instructions provided; Collect blood & Fecal samples (SCR_2.0), aliquot samples; Ship one aliquot of blood and fecal samples to ISU VDL (GBI BI Submission form- individual animal ID labels); Keep vaccine retention samples; Observe injection site lesions for 3 consecutive days (ISX 1.0) |
| Day 7 | | Terminate antibiotic in feed for EIIMATB group (BPT 1.0) |
| Day 28 | | Challenge all animals with Lawsonia (CDR_1.0); Weigh All Animals (BWR_1.0); Collect sample of challenge material pre and post challenge (SCR_2.0); Perform daily clinical observations until end of study (COR_1.0); Collect blood & Fecal samples (SCR_2.0), aliquot samples; Ship one aliquot of blood and fecal samples to ISU VDL (GBI BI Submission form- individual labels) |
| Day 35 | | Collect blood & Fecal samples (SCR_2.0), aliquot samples |
| Day 42 | | Collect blood & Fecal samples (SCR_2.0), aliquot samples |
| Day 49 | | Collect blood & Fecal samples (SCR_2.0), aliquot samples; Weigh all animals (BWR_1.0); Perform Necropsy on all animals; Score and record gross lesions (ONP_2.0); Collect intestinal lavage, scrapping and mesenteric lymph node (SCR 2.0) |
| | | Submit samples UMN for histology (SCR_2.0) for scoring (HIR 2.0) |

### Treatments by Group

The treatment groups were: positive challenge control (PC); Enterisol® Ileitis given IM with Ingelvac®3FLEX vaccine (EIIM) and Enterisol® Ileitis given IM with Ingelvac®3FLEX vaccine with concomitant antimicrobial administration (EIIMATB). A negative control (NC) group of 16 pigs was not challenged, and served the purpose of evaluating the severity of challenge in the PC group. Treatment group EIIMATB was the only group that received antimicrobials and was given the label dosage of 35 g/ton of Denagard® (tiamulin) and 400 g/ton of CTC (chlortetracycline) for a total period of two weeks. After one week of Denagard CTC in feed, the EIIMATB group was vaccinated IM with a 2ml dose of Enterisol® Ileitis combined with Ingelvac®3FLEX vaccine. The lyophilized form of Enterisol® Ileitis vaccine was rehydrated using the Ingelvac®3FLEX vaccine in a manner that resulted in one 2 mL dose of the Enterisol® Ileitis in each 2 mL dose of Ingelvac®3FLEX. This means that a 2 mL dose of the resulting experimental “4FLEX” vaccine contained the appropriate amounts of all four antigens.

### Treatment Dosing

The vaccines were administered to groups 1-5 on D0 via the route indicated in in the below table. Administration of IM injections was performed into the right side of the neck, midway between the base of the ear and point of the shoulder, using appropriately-sized sterile needles and syringes.

Treatments

| | |
|---|---|
| Group | Treatment |
| EIIM | Ingelvac®3FLEX vaccine + Enterisol® Ileitis (IM) [4FLEX + no med] = non-medicated feed/water (EIIM) |
| EIIMAT B | Ingelvac®3FLEX vaccine + Enterisol® Ileitis (IM) [4FLEX + Tiamulin/CTC] = medicated feed (35 ppm Tiamulin, 400 ppm Chlortetracycline) (EIIMATB) |
| PC | Ingelvac®3FLEX vaccine (IM), Challenged [Positive Challenge Control] = non-medicated feed/water (PC) |
| NC | Ingelvac®3FLEX vaccine (IM), Not challenged [Negative Challenge Control] = non-medicated feed/water (NC) |

Challenge

| | |
|---|---|
| Description | Lawsonia intracellularis (LI) gut homogenate |
| Formulation | The challenge consisted of gut homogenate material. A frozen stock of gut homogenate was thawed and diluted in DMEM media immediately prior to challenge. |
| Dosage | The LI challenge dose was determined to achieve a significant high level of lesions and characteristic disease in the positive challenge control group. The dose used was of 8×10⁹ Lawsonia intracellularis organisms per pig, quantified by qPCR at ISU VDL. |
| Challenge Procedure | On D28 all pigs in groups 1-4, received the challenge material by gastric gavage. The challenge was recorded on the Challenge Dosing Record |
| Testing | Challenge material was submitted for shotgun metagenomic sequencing to investigate its complete content. The pathogens of interest Salmonella, Brachyspira and PRRSV were not detected in this material. Quantification of Lawsonia in challenge material was performed by ISU VDL quantitative PCR. |

### Animal Inclusion/Exclusion Criteria

Prior to the initiation of the study each animal’s health was assessed and an animal health examination form was completed. Only animals of normal health status that were qPCR and seronegative for Lawsonia were included in this trial. If welfare or illness concerns arose during the study, the primary investigator and site veterinarian and/or designee determined the best course of action including euthanasia, these events were recorded.

Experimental Unit: The pig was the experimental unit.

### Randomization

The randomization of pigs to pen and treatment was conducted by statistical service provided by GBI using SAS statistics software. Prior to the start of the study, the available pigs, litter information, gender, age, weight, and housing facility set-up was provided to the GBI Statistician. Animals were blocked per weight, sex and litter, a random number generator was be used to assign pigs per treatment.

### Blinding Criteria

Personnel involved with scoring lesions and performing laboratory assays were blinded to the allocation of pigs to groups throughout the study. All study site personnel involved with collecting data for the study completed an entry on the signature record for documentation purposes.

### Veterinary Care and Concomitant Treatment

Following arrival at the study site, no pharmaceuticals were administered without prior consent from the monitor or a designee. The animals were under veterinary supervision upon arrival at the facility until the end of the study. Any animals exhibiting injury or illness unrelated to challenge administration would have been given appropriate veterinary care. Documentation provided by the investigator would include a description of the observed clinical signs, the outcome of any diagnostic examination, and the outcome of any administered treatments. All treatments would have been documented on the Biological and Pharmaceutical Treatment Record, these included the antimicrobial given to the EIIMATB group. Euthanized or expired animals would have been necropsied and samples collected as needed to establish a diagnosis, in the event of this occurrence a veterinary report record would have been completed. Any unexpected illness or moribund animal would have been handled appropriately by the site investigator, site veterinarian in concert with the monitor and PI to determine the best plan to alleviate pain/distress either by treatment or euthanasia.

### General Observations

Beginning at arrival and continuing until D28, all pigs were observed daily for general health and the observations were recorded on a General Health Observation Record.

### Injection Reactions

Pigs were monitored 4-8 hours after vaccination for reactivity at the injection sites by having injection sites observed as well as any adverse events. Pigs were re-evaluated at 1, 2 and 3 days after vaccination. Any animal with an abnormality noted at the injection site would have been monitored daily until the lesion resolved. Abnormalities were determined to be noted and described in terms of size (cm), redness, swelling, heat, and pain. Injection site observations were documented on the Injection Site Observation Record.

### Clinical Observations

All pigs were observed daily from D28 through D49 for clinical signs associated with the Lawsonia intracellularis challenge. The findings were recorded on the Clinical Observations Record using the observations assessment shown in the below table.

Clinical observations assessment

| | | | |
|---|---|---|---|
| Clinical Signs | Diarrhea | Behavior | Body Condition |
| 0-normal | normal feces | Normal behavior | normal body condition |
| 1-mild | semi-solid, no blood | slight to moderately depressed, will stand | thin relative to body frame |
| 2-moderate^{∗} | watery, no blood or dark feces | depressed or recumbent | ribs and backbone pronounced |
| 3-severe^{∗} | blood tinged feces, loose or formed | | |

^{∗}Contact investigator or monitor to determine if pig requires euthanasia for humane reasons

### Body Weights

Each pig was weighed (lb) as indicated in the schedule of events and the results recorded on a Body Weight Record. Average daily weight gain for all test animals was calculated for group comparisons.

### Necropsy

On D49, all remaining pigs were euthanized according to site procedures. The ileum, jejunum, caecum, and colon were examined for gross lesions with the scores being recorded on the Off Test Necropsy Record, following the scoring scheme found in the below table.

Macroscopic Lesion Scoring Scale

| Score | Description |
|---|---|
| 0 | Normal |
| 1 | Mild thickening |
| 2 | Moderate thickening/inflammation |
| 3 | Severe thickening/inflammation |
| 4 | Severe thickening/inflammation/bloody content/clots |
| 5 | Necrosis |

Microscopic Lesion Scoring Scale

| Score | IHC for Lawsonia | Histology (hematoxylin and eosin stains) |
|---|---|---|
| 0 | No foci of organisms | No lesion development |
| 1 | One foci of organisms | Few areas of mild lesion development |
| 2 | Multiple foci of organisms | Moderate multifocal area of moderate lesion development |
| 3 | Diffuse foci of organisms | Diffuse areas of severe lesion development |

### Blood Sample Collection

Blood collection was documented on the Sample Collection Record. Venous whole blood was collected in serum separator tubes (SST) according to the schedule of events. Venous whole blood was collected by the Investigator or designee via the anterior vena cava from each pig using a sterile 18-20 g × 1-1.5” Vacutainer® needle, a Vacutainer® needle holder, and 9 or 13 ml SST tubes. Each sample was labeled with the animal’s ID number, the study number, the date of collection, the study day and the sample type. The SST was allowed to clot at room temperature before centrifugation at approximately 2000xg for 5-10 minutes. Once centrifugation was completed serum aliquots were made for testing and long term storage at -80 C. An aliquot of the serum stored at -80 C was submitted on dry ice to standardize handling of all samples to a diagnostic laboratory (ISU-VDL) for Lawsonia ELISA testing.

### Fecal Sample Collection

Fecal samples were collected by digital insertion into the rectum according to the schedule of events to test for Lawsonia shedding by qPCR at ISU-VDL. Fecal collection was documented on the Sample Collection Record. Fecal samples were collected into tubes labeled with study number, the date of collection, day of study, and pig ID number. All samples were aliquoted in to two tubes and stored at -80° C. One aliquot was shipped in an insulated container with dry ice to maintain samples frozen during shipping and ensure all samples would be treated equally at ISU-VDL.

Collected samples were documented on a Sample Collection Record. Microscopic lesions evaluation was performed by hematoxylin and eosin stains (H&E) and Lawsonia immunohistochemistry (IHC). Test procedure and results are included in this final report; the raw data was recorded using the Histology and Immunohistochemistry Record. Samples of the ileum were collected and formalin fixed for microscopic histological examination at with the scoring being done by a previously established method using the Histology and Immunohistochemistry Record.

### Statistical Analysis

The statistical test followed those used in peer-reviewed publications with similar data and allows for pairwise comparisons. Qualitative data was assessed with the chi-square test. Significance was determined when p < 0.05 and a tendency when 0.05 ≤p < 0.10.

### General Observations/Adverse Events

Three adverse events occurred during the study. No adverse events involved vaccination and one event was related to Lawsonia intracellularis challenge. These events are described in the below table.

General Observations/Adverse Events

| | | |
|---|---|---|
| Adverse Event | Affected Pig/ Group | Study Days Affected |
| Lame/Euthanized | PC | D11 |
| Rectal Prolapse/Euthanized | EIIMATB | D36 |
| Severe Ileitis/Found Dead | PC | D45 |

### Injection Reactions

None of the animals that were vaccinated developed any redness, swelling, heat or pain in the 3 days they were evaluated following vaccination. No difference in injection site reaction was noted in the groups that received Ingelvac®3FLEX vaccine IM alone or when mixed and injected in combination with Enterisol® Ileitis.

### Clinical Observations

Clinical diarrhea scores were of zero or normal in all animals at the time of challenge and began to alter in different groups at 6 days post infection (dpi). The incidence of altered diarrhea score was significantly (p<0.0001) greater in the PC group as compared to the NC group (see below table), indicating that L. intracellularis challenge led to characteristic clinical signs. All groups that received Enterisol® Ileitis had a significant reduction on the incidence of altered clinical diarrhea score as compared to the non-vaccinated PC group (see below table). Behavior was not very much altered following challenge, only one event of altered behavior was noted among 501 evaluations in the PC group. Body condition score was found to be altered due to challenge. All treatment groups had significantly more occurrences of altered body condition as compared to the negative control group (see below table). The EIIM treatment group was the one with the lowest incidence of altered body condition, with a tendency (p=0.054) toward reduced body condition as compared to the non-vaccinated PC group.

Evaluation of clinical scores among treatment groups

| | Diarrhea Score | Behavior Score | Body Condition Score |
|---|---|---|---|
| EIIM | 172/528^{c} | 0/528 | 6/528^{b} |
| EIIMATB | 172/514^{c} | 0/514 | 10/514^{bc} |
| NC | 5/352^{b} | 0/352 | 0/352^{a} |
| PC | 229/501^{a} | 1/501 | 14/501^{bc} |

### Gross Lesion Scores

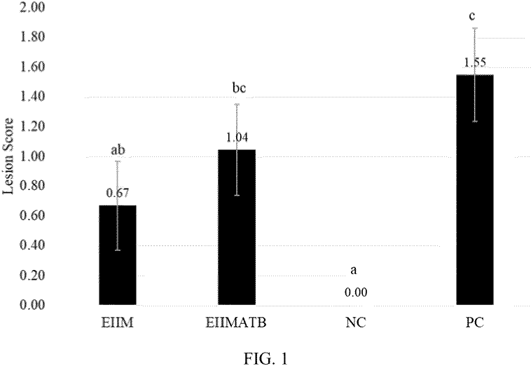

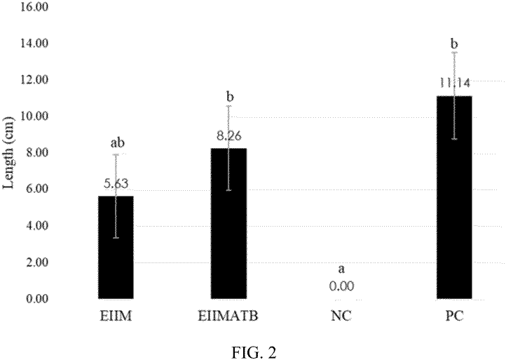

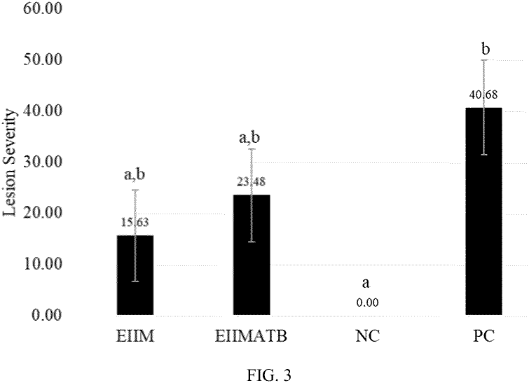

The gross lesion scores in the ileum, the preferential site of L. intracellularis colonization, were highest in the PC group with an average score of 1.55 (FIG. 1). This score was significantly higher than the non-challenged control group (NC), validating this challenge model (FIG. 1, p<0.05). Among vaccinated groups, the EIIM group had the lowest lesion scores with an average score of 0.67 which was significantly (p<0.05) less than the non-vaccinated PC group (FIG. 1). The EIIMATB group developed a similar level of lesions, which were less severe than the PC group but did not reach statistical significance. The length of gross lesions was also measured and followed a similar pattern to that of lesion score. The EIIM group had an average ileum lesion length of around half that the of the PC group (p=0.097) (FIG. 2). A severity score was assigned by multiplying lesion score to lesion length. The average lesion severity scores can be found in FIG. 3. The severity score followed a similar trend to that of lesion score and length. The EIIM group had an average severity score of 15.63, while the PC group had an average score of 40.68 (p=0.053; FIG. 3). Vaccinated groups also led to a reduction of lesions in other portions of the intestinal tract. This was not investigated further since there was not a significant difference in lesions between PC and NC groups, which was likely caused by the fewer number of animals with lesions outside of the ileum.

### Body Weights

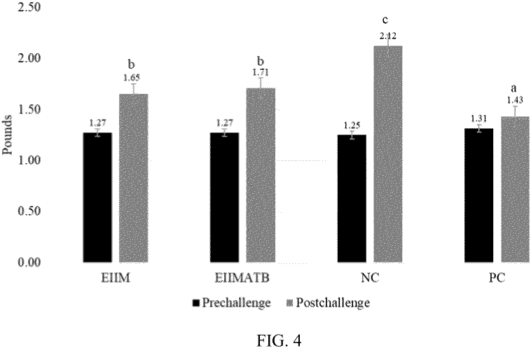

Average daily weight gain (ADG) was very similar among treatment groups in the pre-challenge study period, and no significant differences were found between groups. In the post-challenge period, all groups that received challenge had a significant reduction in ADG as compared to the NC group which did not receive challenge (FIG. 4). The EIIMATB group was the vaccinated group that performed the best in the post challenge period with an ADG of 1.71 lbs/day which was significantly higher than the non-vaccinated challenged PC group (p<0.05) which had an average ADG of 1.43. The EIIM group was the vaccinated group that had the second highest post-challenge ADG which was of 1.65, also significantly higher than the PC group (p<0.05); (FIG. 4).

### Lawsonia Serum ELISA

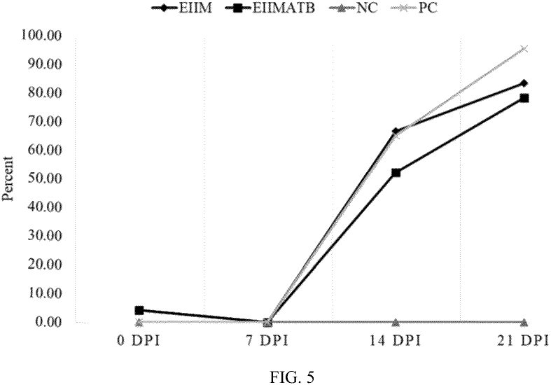

The number of animals with serum antibodies against Lawsonia as measured by ELISA at ISU-VDL is shown in FIG. 5. All animals were seronegative at time of vaccination, study day 0 (28 days prior to challenge, -28 dpi). Only two animals were seropositive at time of challenge (study day 28), one animal in the EIIM group and another in the EIIMATB group. This indicates that IM vaccination of Enterisol® Ileitis in combination with Ingelvac®3FLEX vaccine does not lead to very much seroconversion. As expected, most animals seroconverted and were positive at 21 dpi, also confirming the validity of the Lawsonia challenge which followed the expectations of this challenge model. The PC group had the highest number of animals with anti-Lawsonia antibodies at 21 dpi with 95% of pigs positive. At 14 dpi the EIIM and PC groups had 67% and 65% percent of seropositive pigs, respectively.

### Lawsonia Fecal qPCR

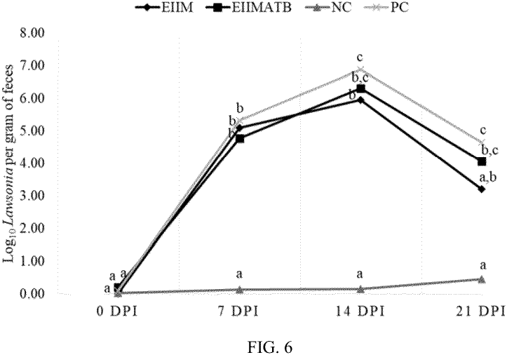

All animals were fecal qPCR negative for Lawsonia at time of vaccination, study day 0 (28 days prior to challenge, -28 dpi). At time of challenge, two animals in the EIIMATB group had detectable levels of Lawsonia in their feces by qPCR. One of the animals in the PC group had a Ct value of 34.1 or 252/organisms per gram of feces at time of challenge. Shedding of Lawsonia peaked at 14 dpi, at which time the PC group shed an average of 6.88 log₁₀ organisms per gram of feces. At this time point, the EIIM group led to a significant (p<0.05) reduction in the fecal shedding compared to the PC group, with a shedding level of 5.96 log₁₀ organisms per gram of feces, respectively. At 21 dpi, the EIIM group was the group that shed the least Lawsonia among challenged animals, with an average of 3.20 log₁₀ organisms per gram of feces. This was significantly less than the non-vaccinated PC group which shed an average of 4.64 log₁₀ organisms per gram of feces (FIG. 6). The EIIMATB group also decreased shedding in comparison to the PC although to a lesser extent with an average of 4.05 log₁₀ organisms per gram of feces, respectively (FIG. 6).

### Microscopic Lesions and Lawsonia IHC

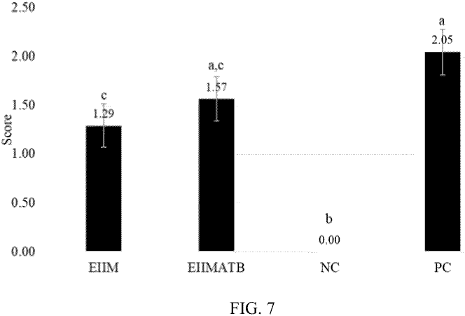

Microscopic lesions were measured by hematoxylin and eosin (H&E) staining of terminal ileum tissue collected at necropsy. The group that developed most severe lesions was the PC group with an average lesion score of 2.05, animals in the NC group did not develop lesions, again validating this infection model (FIG. 7). The vaccinated group that developed least severe lesions was the EIIM group with an average score of 1.29 being significantly reduced from PC group (FIG. 7, p<0.05). The EIIMATB group had a similar but higher average lesion score as compared to the EIIM group with an average lesion score of 1.57.

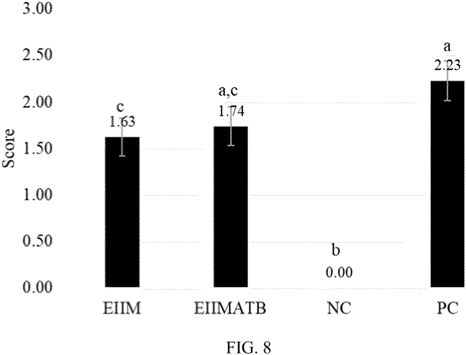

Terminal ileum tissue collected at necropsy and fixed in formalin was also submitted for immunohistochemistry (IHC) staining for L. intracellularis antigen. Similar to HE staining, the group with the highest score was the PC group with an average score of 2.23 being significantly (p<0.05) increased as compared to the NC group, again validating infection and reproduction of disease in this study (FIG. 8). Similar to HE score, again the EIIM group was the vaccinated group with the lowest severity score, with a score of 1.63 being significantly reduced as compared to the PC group (p<0.05).

### Discussion

This study investigated the efficacy of the lyophilized presentation of Enterisol® Ileitis when combined with 3FLEX® vaccine in one 2 ml dose administered by the intramuscular route. Additionally, the interference of tiamulin and chlortetracycline when provided in feed during vaccination was evaluated.

Porcine proliferative enteropathy (PPE), caused by L. intracellularis, was successfully reproduced in this study as measured by characteristic macroscopic lesions, microscopic lesions, fecal shedding of L. intracellularis, seroconversion to L. intracellularis, clinical signs and impact on production performance. The intramuscular combination of Enterisol® Ileitis with 3FLEX®, “4FLEX”, led to a meaningful and significant reduction in macroscopic lesion severity, microscopic lesion severity, clinical diarrhea score and fecal shedding of L. intracellularis. A non-significant but numerical increase in average daily weight gain was also observed in this treatment group (EIIM) compared to non-vaccinated controls (PC). These results indicate that the intramuscular route and mixing of Enterisol® Ileitis with 3FLEX® vaccine are a suitable and efficacious option for the prevention of PPE. It was also observed that this vaccine combination was safe and no adverse events or injection site reactions were observed.

When antimicrobials were administered during vaccination, a change was noted to the parameters evaluated in this study. While a reduction in macroscopic and microscopic lesions was observed in the EIIMATB group relative to non-vaccinated challenged controls, these levels did not reach statistical significance and were numerically increased as compared to the same treatment without antimicrobials (EIIM). The EIIMATB group, however was the vaccinated group with the greatest numerical weight gain following challenge (FIG. 4) and had a significant reduction in the incidence of altered diarrhea clinical score (Evaluation of clinical scores among treatment groups table). These results indicate that antimicrobials may interfere with vaccine efficacy, yet some level of protection was still conferred when vaccinating in the presence of the tested antimicrobials that are effective against Lawsonia.

Nogueira M. G. et al. (Immunological responses to vaccination following experimental Lawsonia intracellularis virulent challenge in pigs. Vet Microbiol. 2013), also found that intramuscular administration of Enterisol® Ileitis led to a reduction of microscopic lesions and fecal shedding of L. intracellularis when compared to non-vaccinated and challenged animals. In that study, the live L. intracellularis antigen present in Enterisol® Ileitis was administered alone, without any adjuvant. The mixing of Enterisol® Ileitis with 3FLEX® could perhaps improve the immune response to the vaccine due to the inclusion of the ImpranFLEX® adjuvant.

This study provides strong evidence that intramuscular administration of Enterisol® Ileitis combined with Ingelvac 3FLEX® offers significant protection against L. intracellularis.

The potential interference of antimicrobials administered during vaccination is not clear and warrants further investigation. However, intramuscular vaccination concomitant with antimicrobial administration did provide a meaningful level of protection in several relevant parameters of disease.

### Example 3: Investigation of Enterisol® Ileitis Given Intramuscularly and With the Presence of Feed Grade Antimicrobials

### Introduction

Enterisol® Ileitis vaccine, is a highly effective, successful product approved for use in healthy post-weaning pigs for the prevention of porcine proliferative enteropathy due to Lawsonia intracellularis to be administered via the drinking water, or via oral drench. No data exists on the efficacy of this product administered via intramuscular injection when combined with 3FLEX® and when administered in conjunction with concomitant antimicrobial therapy.

The objectives of this Example are to 1. Evaluate the efficacy of Enterisol® Ileitis vaccine when combined with 3FLEX® vaccination and administered via intramuscular injection and 2. Evaluate the efficacy of this vaccine combination when administered to pigs receiving concomitant in-feed antimicrobial treatment.

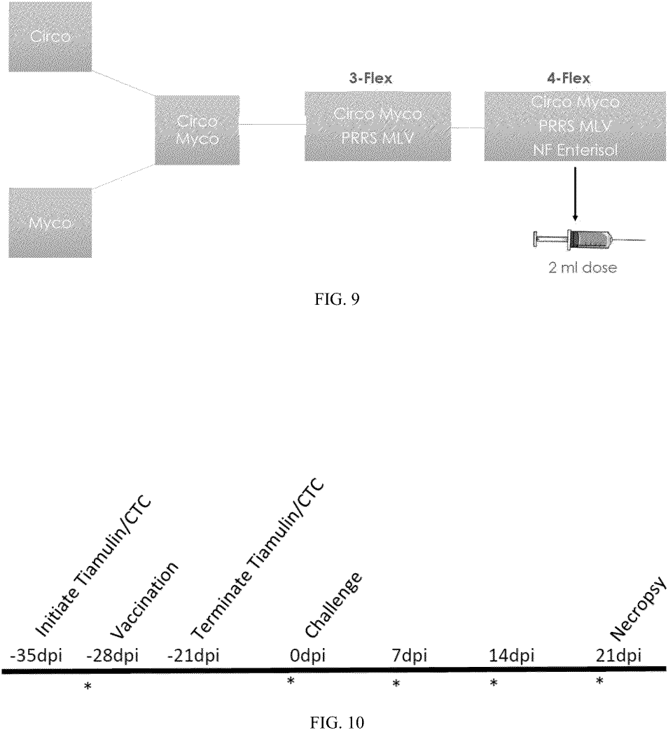

FIG. 9 illustrates vaccine blending.

FIG. 10 provides a study outline.

Primary measured parameters include macroscopic lesion scores, microscopic lesion scores and fecal shedding. Secondary measured parameters include average daily gain, clinical scores and seroconversion.

### Materials and Methods

Pigs were obtained post-weaning at 17-21 days of age and randomly divided into 3 treatment groups with 24 animals each. The treatment groups were: non-vaccinated positive challenge control (PC); Enterisol® Ileitis given IM with 3®FLEX vaccine (EIIM); Enterisol® Ileitis given IM with 3®FLEX vaccine with concomitant antimicrobial administration (EIIMATB). Vaccine preparation was done by reconstituting the lyophilized form of Enterisol® Ileitis with 3FLEX® vaccine. This resulted in a final 2 ml dose containing modified live Lawsonia intracellularis antigen along with PCV2, M. hyo. and PRRSV MLV vaccine fractions. To investigate if in-feed antimicrobial treatment would inhibit the efficacy of vaccination with the live modified Enterisol® Ileitis vaccine, the EIIMATB group received in-feed tiamulin (35 ppm) and chlortetracycline (400 ppm) beginning one week prior to vaccination, and continuing through one week post-vaccination. All animals were challenged with a gut homogenate containing virulent Lawsonia intracellularis 28 days post vaccination and necropsied 21 days later. Clinical signs, average daily weight gain (ADG), fecal shedding as well as macroscopic and microscopic lesions were evaluated at necropsy.

### Results

Both vaccinated treatment groups led to a significant reduction in diarrhea clinical score (P<0.05). Both vaccinated groups also led to an increase in ADG following challenge. The challenge control group had a post challenge ADG of 1.43 lbs while the EIIM and EIIATB groups had ADGs of 1.65 and 1.71 lbs (P<0.05), respectively. Macroscopic lesions scores were decreased in both the vaccinated groups with values of 0.67 and 1.04 compared to the non-vaccinated group which had an average score of 1.55. Similarly, microscopic lesions were also decreased by vaccination with an average score of 2.05 in the PC group compared to averages of 1.29 and 1.57 in the EIIM and EIIMATB groups, respectively. No adverse reactions were noted as the IM vaccine combination was found to be safe.

### Conclusions

The two injectable vaccine groups compared to the non-vaccinated group following Lawsonia challenge improved ADG, led to a reduction of macroscopic and microscopic lesion scores, reduced the fecal shedding of Lawsonia and reduced clinical signs. No evidence of antimicrobial interference was observed with the combination of Enterisol® Ileitis and 3FLEX® intramuscular vaccine. This study demonstrates a new tool for swine producers that warrants further investigation.

### Example 4: Efficacy of Porcine Circovirus Type 2a (PCV2a) ORF2 VLP Vaccine in Combination With Lawsonia ALC Vaccine Administered Intramuscularly

The obj ective of the study is to demonstrate efficacy of Porcine Circovirus Type 2a (PCV2a) ORF2 VLP vaccine (Ingelvac CircoFLEX®) in combination with Lawsonia ALC (avirulent live culture) vaccine (Enterisol® Ileitis, lyophilized) administered intramuscularly in three-week-old pigs against Porcine circovirus Type 2a challenge four weeks later.

Pigs are randomized upon enrollment in this study. During the vaccination phase, pigs are penned with litter mates. Referencing Table below, on Day 0, pigs are 21 ±3 days old at time of vaccination. The PCV2a ORF2 VLP vaccine (Ingelvac CircoFLEX®) is combined with L. intracellularis ALC (avirulent live culture; Enterisol® Ileitis, lyophilized), the Enterisol Ileitis lyophilisate is reconstituted with Ingelvac CircoFLEX (Group 1). Group 2 (Lawsonia avirulent live culture, monovalent) comprises avirulent Lawsonia as well as saline and the adjuvant Carbopol since Carbopol and saline is present in Ingelvac CircoFLEX® and, therefore, Carbopol and saline is also present in groups 1 and 3. Group 3 (PCV2 VLP, monovalent) comprises the PCV2a ORF2 VLP. The NTX group consists of six pigs to serve as non-treatment control.

The pigs are observed for any adverse reactions to the inoculation, including injection site reactions and anaphylaxis.

Lawsonia ALC (avirulent live culture; Enterisol® Ileitis, lyophilized) and PCV2a ORF2 VLP (Ingelvac CircoFLEX®) are registered and well-known veterinary vaccines. However, WO2006/072065 and WO2008/076915 describe the generation of the PCV vaccine, its formulation and its administration. WO 96/39629 and WO 05/011731 describe the cultivation of Lawsonia intracellularis, attenuated Lawsonia intracellularis and its administration.

Prior to challenge (D27), pigs are commingled. NTX Group is necropsied on D20 to establish that pigs are not exposed to wild- type PCV2 during the vaccination phase. On Day 28, pigs are challenged with virulent PCV2a (4.77 log₁₀TCID₅₀/ 2 mL dose) by means of intramuscular injection and intranasal administration. Clinical scores are assigned starting on Day 27 based on observations of fecal consistency, body condition and behavior for the rest of the study.

### Necropsy

Twenty-two days post-challenge animals in groups 1-3 are euthanized and necropsied. Lymph nodes and ileum are assessed, scored, and collected for histopathology and immunohistochemistry.

Experimental Design

| Group | Description | Number of pigs | Vaccination Dose Vol (D0) | VX Route | Challenge (D28) | Necro psy |
|---|---|---|---|---|---|---|
| 1 | Lawsonia avirulent live culture + PCV2 VLP (Associated Use, bivalent) | 24 | 1 mL | IM | D28 PCV2a 1 mL IN + 1 mL IM | D50 |
| 2 | Lawsonia avirulent live culture (monovalent) | 24 | 1 mL | IM | | |
| 3 | PCV2 VLP (monovalent) | 24 | 1 mL | IM | | |
| NTX | NTX strict controls | 6 | n/a | n/a | n/a | D20 |

A general examination of all organs and the injection site region is completed during the necropsy process for abnormalities. Samples of tonsil, trachealbronchial lymph node (TBLN), mesenteric lymph node (MLN), external iliac lymph node (ILN), and ileum are collected and fixed in 10% neutral buffered formalin. Samples are processed in accordance with standard procedures and evaluated by hematoxylin and eosin staining (H&E) for histopathology and by immunohistochemistry (IHC) for PCV2 antigen. Scoring of tissues are conducted following the scoring system below.

Tissue Scoring

| | | |
|---|---|---|
| Tissues | Parameter | Scoring System |
| Tonsil, TBLN, MLN, ILN, Ileum | Lymphoid Depletion | 0 = Normal, no lymphoid depletion present (Negative) |
| | | 1 = Mild lymphoid depletion with loss of overall cellularity (Positive) 2 = Moderate lymphoid depletion (Positive) |
| | | 3 = Severe lymphoid depletion with loss of lymphoid follicle structure (Positive) |
| Tonsil, TBLN, MLN, ILN, Ileum | PCV2 Lymphoid Colonization (IHC) | 0 = Zero lymphoid cells with PCV2 antigen staining (Negative) |
| | | 1 = <10% of lymphoid follicles have cells with PCV2 antigen staining (Positive) |
| | | 2 = 10% to 50% of lymphoid follicles contain cells with PCV2 antigen staining (Positive) |
| | | 3 = >50% of lymphoid follicles contain cells with PCV2 antigen staining (Positive) |

### Lymphoid Depletion

A pig is considered positive if one or more of the four lymphoid tissue samples (tonsil, TBLN, MLN, ILN) or ileum is histologically positive (score > 0) for lymphoid depletion.

### Lymphoid Colonization

A pig is considered positive if one or more of the four lymphoid tissue samples (tonsil, TBLN, MLN, ILN) or ileum is positive (score > 0) for PCV2 lymphoid colonization by IHC.

### Tissue Results (Histopathology and Immunohistochemistry)

Results presented in the table below represent any tissue that had a positive score as described in the Tissue Scoring table (score of 1, 2, 3). Percentages reflect the number of pigs with a positive score out of the total number of pigs in the group.

| | Lymphoid depletion | | | | | PCV2a IHC | | | | |
|---|---|---|---|---|---|---|---|---|---|---|
| | LD_TBLN | LD_MLN | LD_EILN | LD_ Tonsil | LD_ Ileum | IHC_TBLN | IHC_MLN | IHC_EILN | IHC_ Tonsil | IHC_ Ileum |
| NTX | 0% | 0% | 0% | 0% | 0% | 0% | 0% | 0% | 0% | 0% |
| T01 | 0% | 0% | 0% | 0% | 0% | 0% | 0% | 0% | 0% | 0% |
| T02 | 88% | 75% | 92% | 79% | 75% | 96% | 88% | 100% | 100% | 88% |
| T03 | 0% | 0% | 0% | 0% | 0% | 0% | 0% | 0% | 0% | 0% |

The results in group 2 show that the challenge was successful because animals only vaccinated with the avirulent live Lawsonia vaccine show high incidence of infection with PCV2. Further, the results show that the PCV2 vaccine is efficacious in combination with avirulent live Lawsonia vaccine since group 1 does not show any clinical signs of PCV2 infection. Group 1 (Lawsonia avirulent live culture + PCV2 VLP; bivalent vaccine) behaves as Group 3 (PCV2 VLP, monovalent vaccine) which was vaccinated with the PCV2 vaccine only. Thus, no interference is observed when combining the PCV2 vaccine with the avirulent live Lawsonia vaccine.

What is claimed is:

1. A vaccine comprising an antigen of Lawsonia intracellularis and one or more antigens of at least one further pathogen selected from the group of porcine circovirus (PCV), Mycoplasma hyopneumoniae (M. hyo.) and porcine respiratory and reproductive syndrome virus (PRRSV), wherein the antigen of Lawsonia intracellularis is live Lawsonia intracellularis.

2. The vaccine of claim 1, wherein the further pathogen is PCV.

3. The vaccine of claim 1, wherein the further pathogen is M. hyo.

4. The vaccine of claim 1, wherein the further pathogen is PRRSV.

5. The vaccine of claim 1, wherein the further pathogens are PCV and M. hyo.

6. The vaccine of claim 1, wherein the further pathogens are PCV and PRRSV.

7. The vaccine of claim 1, wherein the further pathogens are PRRSV and M. hyo.

8. The vaccine of claim 1, wherein the further pathogens are PCV, M. hyo and PRRS.

9. The vaccine of any one of claims 1, 2 5, 6 and 8, wherein the antigen of PCV is a recombinant polypeptide.

10. The vaccine of claim 9, wherein the recombinant polypeptide is expressed by a PCV ORF gene.

11. The vaccine of claim 10, wherein the PCV ORF gene is a PCV ORF2 gene.

12. The vaccine of any one of claims 9 to 11, wherein the antigen is expressed in a baculovirus cell.

13. The vaccine of any one of claims 1, 2, 5 and 6, wherein the antigen of PCV is the antigen included in Ingelvac CircoFLEX®.

14. The vaccine of any one of claims 1, 2, 5, 6 and 8 to 13, wherein the vaccine has a dosage of about 2 µg to about 400 µg of the antigen of PCV or a dosage of about 2 µg to about 400 µg of the PCV2 ORF2 protein.

15. The vaccine of any one of claims 1, 3, 5 and 7, wherein the antigen of M. hyo. is a supernatant and/or a bacterin.

16. The vaccine of any one of claims 1, 3, 5, 7 and 15 wherein the antigen of M. hyo. is the antigen included in Ingelvac MycoFLEX®.

17. The vaccine of any one of claims 1, 4, 6 and 7, wherein the antigen of PRRSV is a live PRRSV virus.

18. The vaccine of claim 17, wherein the live PRRSV virus is a modified live virus.

19. The vaccine of claim 17, wherein the live PRRSV virus is an attenuated virus.

20. The vaccine of any one of claims 17 to 19, wherein the vaccine has a dosage of the antigen of PRRSV of about 10¹ to about 10⁷ viral particles per dose or about 10⁴ to about 10⁷ particles per dose.

21. The vaccine of any one of claims 1, 4, 6, 7 and 17 to 20, wherein the antigen of PRRSV is lyophilized.

22. The vaccine of any one of claims 1, 4, 6, 7 and 17 to 21 wherein the antigen of PRRSV is the antigen included in Ingelvac® PRRSV MLV.

23. The vaccine of claim 1, wherein the antigen of PCV, M. hyo. and PRRSV is the antigen of PCV, M. hyo. and PRRSV included in 3FLEX®.

24. The vaccine of any one of claims 1 to 23, wherein live Lawsonia intracellularis is modified-live Lawsonia intracellularis.

25. The vaccine of any one of claims 1 to 23, wherein live Lawsonia intracellularis is attenuated Lawsonia intracellularis.

26. The vaccine of any one of claims 1 to 25, wherein the vaccine has a dosage of the antigen of Lawsonia intracellularis of about 10³ to 10⁹ bacteria/Kg of body weight, preferably of about 10⁵ to 10⁷ bacteria/Kg of body weight.

27. The vaccine of any one of claims 1 to 26, wherein the vaccine has a dosage of the antigen of Lawsonia intracellularis of about 10⁵ to about 10⁷ of Lawsonia intracellularis bacteria.

28. The vaccine of any one of claims 1 to 27, wherein the antigen of Lawsonia intracellularis is lyophilized.

29. The vaccine of any one of claims 1 to 28, wherein the antigen of Lawsonia intracellularis is the antigen included in Enterisol® Ileitis.

30. The vaccine of any one of claims 1 to 29, wherein the vaccine further comprises one or more adjuvant(s).

31. The vaccine of claim 30, wherein the adjuvant(s) comprise(s) one or more of a polymer of acrylic or methacrylic acid; copolymer of maleic anhydride and alkenyl derivative; a polymer of acrylic or methacrylic acid which is cross-linked; a polymer of acrylic or methacrylic acid which is cross-linked with a polyalkenyl ether of sugar or polyalcohol; a carbomer; an acrylic polymer cross-linked with a polyhydroxylated compound having at least 3 and not more than 8 hydroxyl groups with hydrogen atoms of at least three hydroxyls optionally or being replaced by unsaturated aliphatic radicals having at least 2 carbon atoms with said radicals containing from 2 to 4 carbon atoms such as vinyls, allyls and other ethylenically unsaturated groups and the unsaturated radicals may themselves contain other substituents, such as methyl; a carbopol®; Carbopol® 974P; Carbopol® 934P; Carbopol® 971P; Carbopol® 980; Carbopol® 941P; ImpranFLEX®; aluminum hydroxide; aluminum phosphate; a saponin; Quil A; QS-21; GPI-0100; a water-in-oil emulsion; an oil-in-water emulsion; a water-in-oil-in-water emulsion; an emulsion based on light liquid paraffin oil or European Pharmacopea type adjuvant; an isoprenoid oil; squalane; squalene oil resulting from oligomerization of alkenes or isobutene or decene; (an) ester(s) of acid(s) or of alcohol(s) containing a linear alkyl group; plant oil(s); ethyl oleate; propylene glycol di-(caprylate/caprate); glyceryl tri-(caprylate/caprate); propylene glycol dioleate; (an) ester(s) of branched fatty acid(s) or alcohol(s); isostearic acid ester(s); nonionic surfactant(s); (an) ester(s) of sorbitan or of mannide or of glycol or of polyglycerol or of propylene glycol or of oleic, or isostearic acid or of ricinoleic acid or of hydroxystearic acid, optionally ethoxylated, anhydromannitol oleate; polyoxypropylene-polyoxyethylene copolymer blocks, a Pluronic product, RIBI adjuvant system; Block co-polymer; SAF-M; monophosphoryl lipid A; Avridine lipid-amine adjuvant; heat-labile enterotoxin from E. coli (recombinant or otherwise); cholera toxin; IMS 1314, or muramyl dipeptide.

32. The vaccine of claim 30 or 31, wherein the adjuvant(s) is/are (a) carbomer(s).

33. The vaccine of any one of claims 30 to 32, wherein the adjuvant(s) is/are Carbopol® and/or ImpranFLEX®.

34. The vaccine of any one of claims 1 to 33, wherein live Lawsonia intracellularis is attenuated Lawsonia intracellularis and/or the antigen of PCV is a recombinant polypeptide expressed by a PCV ORF2 gene and/or the antigen of M. hyo. is a bacterin and/or the antigen of PRRSV is an attenuated PRRSV virus.

35. The vaccine of any one of claims 1 and 24 to 34, wherein the antigen of Lawsonia intracellularis is lyophilized and dissolved in the 3FLEX® vaccine.

36. The vaccine of claim 35, wherein the antigen of Lawsonia intracellularis is Enterisol® Ileitis.

37. The vaccine of any one of claims 1 to 36, wherein the vaccine further comprises a pharmaceutically or veterinarily acceptable carrier.

38. The vaccine of any one of claims 1 to 37, wherein the vaccine is in a form for systemic administration.

39. The vaccine of any one of claims 1 to 38, wherein the vaccine is formulated and/or packaged for a single dose or one-shot administration.

40. The vaccine of any one of claims 1 to 38, wherein the vaccine is formulated and/or packaged for a multi-dose regimen, preferably a two-dose regimen.

41. The vaccine of any one of claims 1 to 40, wherein the vaccine is in a dosage form; and wherein said dosage form is delivered from a container containing a larger amount of said vaccine and wherein a dosage form of said vaccine is capable of being delivered from said container.

42. The vaccine of claim 41, wherein the container contains at least 10, at least 50, at least 100, at least 150, at least 200 or at least 250 doses of said vaccine.

43. The vaccine of any one of claims 1 to 42 for use in a method for eliciting a protective immune response in an animal comprising administering said vaccine to the animal.

44. The vaccine for use of claim 43, wherein the animal is a pig.

45. The vaccine for use of claim 43 or 44, wherein the method is for eliciting a protective immune response against Lawsonia intracellularis and/or PCV and/or M. hyo. and/or PRRSV in the animal.

46. The vaccine for use of any one of claims 43 to 45, wherein the vaccine is administered systemically.

47. The vaccine for use of any one of claims 43 to 46, wherein the vaccine is administered as one dose.

48. The vaccine for use of any one of claims 43 to 47, wherein animal is simultaneously/concomitantly treated with one or more antibiotic(s).

49. The vaccine for use of any one of claims 43 to 48, wherein the method is for immunizing an animal against a clinical disease caused by at least one pathogen in said animal, wherein said vaccine fails to cause clinical signs of infection but is capable of inducing an immune response that immunizes the animal against pathogenic forms of said at least one pathogen.

50. The vaccine for use of any one of claims 43 to 49, wherein the protective immune response against Lawsonia intracellularis is for reducing intestinal lesions in an animal, in comparison to an animal of a non-immunized control group of the same species.

51. The vaccine for use of claim 50, wherein the intestinal lesions are ileum lesions.

52. The vaccine for use of claim 50 or 51, wherein the intestinal lesions are macroscopic lesions and/or microscopic lesions.

53. The vaccine for use of any one of claims 43 to 52, wherein the protective immune response against Lawsonia intracellularis is for reducing fecal shedding of an animal, in comparison to an animal of a non-immunized control group of the same species.

54. The vaccine for use of any one of claims 43 to 53, wherein the protective immune response against Lawsonia intracellularis is for increasing the average daily weight gain of an animal, in comparison to an animal of a non-immunized control group of the same species.

55. The vaccine for use of any one of claims 43 to 54, wherein the vaccine is protective against a challenge with 8×10⁹ Lawsonia bacteria.

56. A method for eliciting a protective immune response against Lawsonia intracellularis and/or PCV and/or M. hyo. and/or PRRSV in an animal comprising administering to the animal the vaccine of any one of claims 1 to 42.

57. A method of immunizing an animal against a clinical disease caused by at least one pathogen in said animal, said method comprising the step of administering to the animal the vaccine of any one of claims 1 to 42, wherein said vaccine fails to cause clinical signs of infection but is capable of inducing an immune response that immunizes the animal against pathogenic forms of said at least one pathogen.

58. Use of a vaccine of any one of claims 1 to 42 in the preparation of a composition for inducing a protective immune response against Lawsonia intracellularis and/or PCV and/or M. hyo. and/or PRRSV or for a method for inducing a protective immune response against Lawsonia intracellularis and/or PCV and/or M. hyo. and/or PRRSV.
